(19) United States
(12) Patent Application Publication
(10) Pub. No.: US 2026/0250323 A1
(43) Pub. Date: Aug. 27, 2026

(54) Molecular Weight Standards

(71) Applicant: BioCatalist LLC, Torrance, CA (US)

(72) Inventor: Jamshid Davoodi, Playa Del Rey, CA (US)

(73) Assignee: BioCatalist LLC, Torrance, CA (US)

(21) Appl. No.: 19/543,977

(22) Filed: Feb. 19, 2026

Related U.S. Application Data
(60) Provisional application No. 63/760,466, filed on Feb. 19, 2025.

(51) Int. Cl. C07K 14/00; C07K 1/12; C12N 15/63

(52) U.S. Cl. CPC C07K 14/00; C07K 1/12; C12N 15/63; C07K 2319/50; C07K 2319/95

(57) ABSTRACT

This disclosure concerns a recombinant protein comprising one or more repeating units, each of the one or more repeating units comprising a chemical cleavage site and a molecular weight polypeptide standard. The disclosure also concerns corresponding nucleic acids, vectors and host cells, a method of producing a plurality of molecular weight polypeptide standards and the resultant set of molecular weight standards and use thereof for calibrating a MALDI-TOF mass spectrometer.

This application is a 35 U.S.C. § 111 patent application that claims the benefit of priority and is entitled to the filing date pursuant to 35 U.S.C. § 119 (e) of U.S. Provisional Patent Application 63/760,466, filed Feb. 19, 2025, the content of which is hereby incorporated by reference in its entirety.

Submitted as part of this patent application is a Sequence Listing filed as an XML file named UEBIO3-0002US-SeqList-SD26.xml having a file size of 92,000 bytes and generated on Feb. 20, 2025, the content of which is hereby expressly incorporated by reference in its entirety.

Mass spectrometry has been used for the analysis and characterization of large molecules including biomolecules. One technique that has widely been used for the analysis of biomolecules including nucleic acids, proteins, peptides and carbohydrates is Matrix-Assisted Laser Desorption/Ionization-time-of-flight (MALDI-TOF) mass spectrometry. This technique combines MALDI with TOF mass spectrometry. Israr et al., Clin. Chem. Lab Med. 58 (6): 883-896 (2020) provides a review of the MALDI-TOF techniques and clinical applications thereof, e.g., for analysis of biomolecules in a diagnostic context.

An important element of analysis by mass spectrometry is calibration of the mass spectrometer being used. This is generally achieved using molecular weight standards comprising several molecules having molecular weights which fall within the range of molecular weights likely to arise from ionization of the samples to be analyzed. When mass spectrometry is used to analyze biomolecules, proteins or polypeptides can be used as molecular weight standards.

Known protein molecular weight standards suffer from a number of disadvantages. For example, US 2006/0023808 and WO 2005/114220 describe calibration of mass spectrometers using recombinant protein standards. However, each different protein in the set of molecular weight standards is expressed separately.

Others have expressed concatemers of different artificial polypeptides a single recombinant molecule and subsequently digested them with a protease (typically trypsin) to yield a set of molecular weight standards comprising multiple peptides having different molecular weights. Such methods are described in Briebeck et al., J. Am. Soc. Mass Spectrom. 25 (8): 1489-1497 (2014) and U.S. Pat. No. 9,481,720. However, the proteases used in production of sets of molecular weight standards may be difficult to fully remove, and the resultant standards may exhibit poor storage stability and/or poor crystallization when mixed with the matrix. Such problems are often exacerbated by the hostile conditions provided by the matrix compositions and solvents used in MALDI-TOF.

The subject matter of the present specification generally relates to the provision of molecular weight standards and methods of producing molecular weight standards that aim to overcome one or more of these problems and/or to provide alternative methods for producing molecular weight standards having improved properties. To achieve these results, the present specification provides recombinant protein comprising a plurality of repeating units each including a molecular weight polypeptide standard and chemically cleavable recognition site sequences arranged in a particular manner. A recombinant protein disclosed herein is expressed, isolated and cleaved to produce one or more molecular weight polypeptide standards having defined molecular weights. Accordingly, the present specification discloses to a recombinant protein, nucleic acid molecules encoding the recombinant protein, vectors and host cells. The present specification also discloses methods of producing a plurality of molecular weight polypeptide standards, a set of molecular weight polypeptide standards obtainable by the method disclosed herein, and use of the set of molecular weight standards for calibrating a mass spectrometer, such as a MALDI-TOF mass spectrometer.

### SUMMARY

Aspects of the present specification provide a recombinant protein comprising one or more repeating units, each of the one or more repeating units comprising a chemical cleavage site and a molecular weight polypeptide standard, wherein the chemical cleavage site is an amino acid sequence, the recombinant protein arranged according to formula I

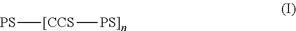

wherein brackets indicate a repeating unit of the one or more repeating units; PP is a molecular weight polypeptide standard; CCS is a chemical cleavage recognition site; and n is an integer from 1 to 50. The disclosed repeating units can comprise two or more different molecular weight polypeptide standards, each having a different amino acid length of a determined molecular weight and each present in one or more copies. Alternatively, or in addition to, the disclosed repeating units can comprise two or more of different molecular weight polypeptide standards, each having the amino acid length but different linear order of amino acids, or different residues, with each having the same determined molecular weight but a different amino acid sequence, and each present in one or more copies. A chemical cleavage site is cleaved by a site-specific proteolytic chemical agent, such as, e.g., 2-nitro-5-thiocyanobenzoic acid (NTCB), Ni²⁺, Cu²⁺, Co²⁺ or Pd²⁺.

Other aspects of the present disclosure provide a recombinant protein discloses herein that may further, and optionally, include a purification tag or a solubilization tag, the recombinant protein arranged according to formula II, formula III, formula IV or formula V:

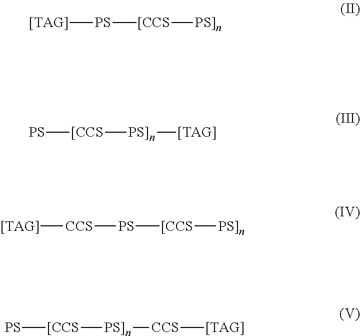

wherein brackets indicate a repeating unit of the one or more repeating units; PP is a molecular weight polypeptide standard; CCS is a chemical cleavage recognition site; TAG is the affinity purification tag or the solubilization tag; and n is an integer from 1 to 50. Depending on the nature of amino acid, e.g., its stability, the TAG may or may not form part of the molecular weight standard. In some embodiments, and as exemplified in formulas II and III, the tag can form part of a molecular wight standard polypeptide. In some embodiments, and as exemplified in formulas IV and V, the tag does not form part of a molecular wight standard polypeptide. A tag may be present at the N-terminus or the C-terminus of the recombinant protein or, in some embodiments, at both the N-terminus and the C-terminus. Addition of a tag or tags may serve to facilitate purification (e.g., affinity purification) and/or solubilization of the protein. In some embodiments, the chemical cleavage site closest to the tag is replaced by a protease cleavage site. For example, the protease cleavage site can be a site recognized and cleaved by a caspase, e.g., caspase-3 or caspase-7. In some embodiments, the protein comprises a sequence selected from SEQ ID NO: 59 or SEQ ID NO: 86.

A further aspect of the present specification provides a nucleic acid molecules encoding the recombinant polypeptide disclosed herein. Nucleic acid molecules refers to a polymeric form of nucleotides (i.e., polynucleotides), including RNA, cDNA, genomic DNA, and synthetic forms and mixed polymers of the above. A nucleotide may refer to a ribonucleotide, deoxyribonucleotide, or a modified form of either type of nucleotide. A nucleic acid molecule can include either or both naturally occurring and modified nucleotides linked together by naturally occurring and/or non-naturally occurring nucleotide linkages.

A further aspect of the present specification provides an expression vector comprising the nucleic acid disclosed herein. A vector may be, for example, of plasmid origin. Vectors contain “replicon” polynucleotides that facilitate the autonomous replication of the vector in a host cell. A vector is typically used to transport one or more polynucleotides into a suitable host cell. Once in the host cell, the vector can replicate independently of or coincidentally with the host chromosomal DNA, and several copies of the vector and its inserted polynucleotide(s) can be generated. In addition, the vector may also contain necessary elements that permit transcription of an inserted polynucleotide into an mRNA molecule, or otherwise cause replication of the inserted polynucleotide into multiple copies of RNA. Some expression vectors additionally contain sequence elements adjacent to the inserted polynucleotide that increase the half-life of the expressed mRNA, and/or allow translation of the mRNA into a protein molecule. Many molecules of mRNA and polypeptide encoded by the inserted polynucleotide can thus be rapidly synthesized.

A further aspect of the present specification provides a host cell comprising the nucleic acid or the vector of the present specification. Recombinant expression methods using many types of host cell are known. In some embodiments, the host cell is selected from a bacterial cell, a fungal cell, an insect cell, a mammalian cell or a plant cell. Bacterial cells include Escherichia coli which has been widely used in the art for recombinant protein expression. Fungal cells include yeast cells, such as Saccharomyces cerevisiae.

Another aspect of the present specification provides a method of producing a plurality of molecular weight polypeptide standards. The disclosed method can comprise: a) expressing a recombinant protein arranged according to formulas I, II, III, or IV disclosed herein; b) isolating the expressed recombinant protein; c) cleaving the chemical cleavage site or sites of the isolated recombinant protein using a chemical agent that recognizes the chemical cleavage site to produce a plurality of molecular weight polypeptide standards, wherein each molecular weight polypeptide standard has its amino acid sequence without any extraneous amino acids. In some aspects, the cleaving of step c) results in partial digestion of the recombinant protein. This may result in two or more repeating units remaining connected and can allow for the provision of longer, higher molecular weight standard proteins without the need to include an individual repeating unit providing a higher molecular weight within the recombinant protein as a whole. This can reduce the overall size of the protein to be expressed. In other aspects, the cleaving of step c) results in complete digestion of the polypeptide.

Another aspect of the present specification provides a set of molecular weight polypeptide standards obtainable by the method disclosed herein.

Another aspect of the present specification provides for use of the set of molecular weight standards disclosed herein for calibrating a MALDI-TOF mass spectrometer. Other uses of the set of molecular weight standards are also envisaged, including for calibration of other types of mass spectrometer

The foregoing and other features will become more apparent from the following detailed description, which proceeds with reference to the accompanying figures.

### BRIEF DESCRIPTION OF THE DRAWINGS

The accompanying drawings, which are incorporated in and constitute a part of this specification, illustrate aspects of the disclosed subject matter in at least one of its exemplary embodiments, which are further defined in detail in the following description. Features, elements, and aspects of the disclosure are referenced by numerals with like numerals in different drawings representing the same, equivalent, or similar features, elements, or aspects, in accordance with one or more embodiments. The drawings are not necessarily to scale, emphasis instead being placed upon illustrating the principles herein described and provided by exemplary embodiments of the invention. More generally, those skilled in the art will appreciate that the drawings are schematic in nature and are not to be taken literally or to scale in terms of material configurations, sizes, thicknesses, and other attributes of an apparatus according to aspects of the present invention and its components or features unless specifically set forth herein. In such drawings:

FIGS. 1A-1B are positive mode MALDI-TOF spectra for exemplary molecular weight polypeptide standards V5 and V6 in linear and reflector modes, with FIG. 1A showing the MALDI-TOF linear mode spectra for molecular weight polypeptide standards V5 and V6 and FIG. 1B showing the MALDI-TOF reflector mode spectra for molecular weight polypeptide standards V5 and V6;

FIG. 2 is a negative mode MALDI-TOF spectra for exemplary molecular weight polypeptide standards V5 in linear negative and reflector negative modes;

FIGS. 3A-3B are the Q-Exactive mass spectra which provide precise determination of the masses of the constituent polypeptides with FIG. 3A showing the exemplary molecular weight polypeptide standards V3 and FIG. 3B showing the exemplary molecular weight polypeptide standards V6;

FIG. 4A-4B are positive mode MALDI-TOF spectra of exemplary standard V2 after exposure to various temperatures for 8 months in linear and reflector modes, with FIG. 4A showing the MALDI-TOF linear mode spectra for standard V2 and FIG. 4B showing the MALDI-TOF reflector mode spectra for standard V2;

FIG. 5 is a positive mode MALDI-TOF spectra of exemplary molecular weight polypeptide standards V5 and V6 on a MALDI plate in the presence of CHCA (α-Cyano-4-Hydroxycinnamic Acid) matrix stored at room temperature for three days; and

FIG. 6A-6B are positive mode MALDI-TOF spectra of exemplary molecular weight polypeptide standards V2 after various freeze-thaw cycles in linear and reflector modes, with FIG. 6A showing the MALDI-TOF linear mode spectra for molecular weight polypeptide standards V2 and FIG. 6B showing the MALDI-TOF reflector mode spectra for molecular weight polypeptide standards V2.

### DETAILED DESCRIPTION

Matrix-Assisted Laser Desorption/Ionization (MALDI) is an ionization technique that uses a laser energy-absorbing matrix to create ions from large molecules with minimal fragmentation. is a process wherein analyte is embedded in a solid or crystalline “matrix” of light-absorbing molecules (e.g., nicotinic, sinapinic, or 3-hydroxypicolinic acid), then desorbed by laser irradiation and ionized from the solid phase into the gaseous or vapor phase and accelerated as intact molecular ions towards a detector. The methodology involves a three-step process where a sample of an analyte is embedded in a suitable solid or crystalline matrix material of light-absorbing molecules and applied to a metal plate. The plated matrix-analyte admixture is then irradiated by a pulsed laser which triggers ablation and desorption of the analyte and matrix material. Finally, the analyte molecules are ionized from the solid phase into the gaseous or vapor phase by being protonated or deprotonated in the hot plume of ablated gases, and then accelerated as intact molecular ions towards a detector of a mass spectrometer. The underlying mechanisms of MALDI ionization methods are described by Knochenmuss, New Developments in Mass Spectrometry 12: 3-19 (2022) and by Harrison, Mass Spectrometry Reviews 16:201-217 (1997) with particular reference to amino acids and peptides.

The matrix material is typically a solution of small organic acid in a solvent. Suitable MALDI matrix materials include α-cyano-4-hydroxycinnamic acid (α-CHCA), 3,5-dimethoxy-4-hydroxycinnamic acid (sinapinic acid or SA), 2,5-dihydroxybenzoic acid (gentisic acid or DHB), 4-hydroxy-3-methoxycinnamic acid (ferulic acid), picolinic acid (PA) and 3-hydroxypicolinic acid (HPA). DHB is a useful matrix for proteins, peptides, DNA and synthetic polymers, whereas α-CHCA is a useful matrix for proteins and peptides, but not DNA because the ionic signal of DNA cannot be detected. SA can be used for proteins and polymers. HPA is a suitable matrix for DNA. For low molecular weight compounds such as metabolites and lipids, 9-aminoacridine is a suitable matrix in the negative ionization mode, whereas trihydroxyacetophenone is useful for the detection of oligonucleotides and phosphorylated peptides. SA, CHCA and DHB are amongst the most commonly used matrices.

Suitable MALDI solvents include a mixture of water with one or more of acetonitrile, methanol, acetone, chloroform, propanol or ethanol. A counter ion source such as trifluoroacetic acid (TFA) is usually added. A typical matrix-solution comprises a matrix compound, solvent and TFA, e.g., 20 mg/ml sinapinic acid in acetonitrile:water:TFA (50:50:0.1). An analyte in typically combined with a matrix material in a 10,000:1 molar ratio of matrix/analyte and can be adjusted to neutral pH before use.

The type of a mass spectrometer most widely used with MALDI is the time-of-flight mass spectrometer (TOF). This method determined an ion's mass-to-charge (m/z) ratio by a time of flight measurement. Ions are accelerated by an electric field of known strength. This acceleration results in an ion having the same kinetic energy as any other ion that has the same charge. The velocity of the ion depends on the mass-to-charge ratio (heavier ions of the same charge reach lower speeds, although ions with higher charge will also increase in velocity). The time that it subsequently takes for the ion to reach a detector at a known distance is measured. This time will depend on the velocity of the ion, and therefore is a measure of its mass-to-charge ratio. From this ratio and known experimental parameters, one can identify the ion.

MALDI-TOF mass spectrometry instruments are often equipped with a reflectron (an “ion mirror”) that uses an electric field to reflect ions and corrects the kinetic energy distribution in the direction of ion flight. This increases the ion flight path, thereby increasing time of flight between ions of different mass-to-charge ratios and increasing resolution. The reflectron uses a constant electrostatic field to reflect the ion beam toward the detector. The more energetic ions penetrate deeper into the reflectron and take a slightly longer path to the detector. Less energetic ions of the same mass-to-charge ratio penetrate a shorter distance into the reflectron and, correspondingly, take a shorter path to the detector. The flat surface of the ion detector (typically a microchannel plate, MCP) is placed at the plane where ions of same mass-to-charge ratio but with different energies arrive at the same time counted with respect to the onset of the extraction pulse in the ion source. A point of simultaneous arrival of ions of the same mass-to-charge ratio but with different energies is often referred to as time-of-flight focus. An additional advantage to the MALDI-TOF/reflectron arrangement is that twice the flight path is achieved in a given length of the TOF instrument.

MALDI-TOF can be carried out in a number of different modes. First, the mode may be linear mode or reflector mode. Linear mode provides higher sensitivity, up to about 10 times higher than reflector mode, but at a lower resolution and is typically used for molecules having a molecular weight larger than approximately 4 kDa. Reflector mode provides lower sensitivity but much higher resolution, such that even different isotopes of the peptides can be detected and distinguished. Reflector mode is typically used for molecules having a molecular weight smaller than approximately 4 kDa. Both linear mode and reflector mode can be operated in positive or negative modes in which the ionized species are protonated (positive mode) or deprotonated (negative mode). Thus MALDI-TOF can be carried out in linear positive, linear negative, reflector positive or reflector negative modes.

Molecular weight polypeptide standards are required to maintain the instrument's mass calibration including evaluation of instrument setup, function and resolution, optimization of instrument parameters including sensitivity, and generation of a mass scale to establish mass accuracy in order to evaluate an unknown analyte or analytes containing a broad range of molecular weights. For MALDI-TOF, such standards allow the translation of a time of flight into a mass of the analyte. As such, it is critical that the polypeptides being used as molecular weight standards for calibration of a mass spectrophotometer

Currently, molecular weight polypeptide standards for MALDI-TOF are produced by chemical synthesis procedures or proteolytic digestion of a protein into smaller polypeptide fragments. However, there are disadvantages to both procedures.

With respect to chemical synthesis protocols, while effective in producing polypeptide standards of small size, these procedures have difficulty in synthesizing polypeptides above 100 residues due to side chain reactions and other inherent constraints of these procedures. As such, chemical synthesis protocols is impractical for producing molecular weight polypeptide standards above 10 kDa. On the other hand, the disclosed molecular weight polypeptide standards and methods of producing such standards can include polypeptide standards as large as 250 kDa.

In addition, chemical synthesis of a polypeptide does not allow for post-translational modifications. The disclosed molecular weight polypeptide standards and methods of producing such standards provide an advantage over chemical synthesis protocols because the expressed recombinant protein can be post-translationally modified. For example, glycosylation sequences can be incorporated into one or more of the molecular weight polypeptide standards to provide glycosylated polypeptides after expression of the recombinant protein.

With respect to existing proteolytic digestion protocols, while enabling the generation of larger molecular weight polypeptide standards, this is achieved at the expense and sacrifice of precision and stability. For example, expression of a recombinant protein comprising concatemers of different artificial polypeptides and subsequently digested them with a protease (typically trypsin) can yield a set of molecular weight standards comprising multiple peptides having different molecular weights. However, non-specific cleavage may adversely affect such use by producing polypeptide fragments of unexpected weight due to such off-target cleavage events. In addition, when preparing molecular weight polypeptide standards for calibration using recombinant biology methods it is essential that the method processes the recombinant protein in a manner that reproducibly produces the intended mass of each weight polypeptide standard because extraneous amino acids no only alter the molecular weight of the standard buy often affect the properties of the resulting polypeptide standard, for example its stability and, in the context of MALDI-TOF, the ability of samples to undergo appropriate crystallization and ionization. The disclosed molecular weight polypeptide standards and methods of producing such standards provide an advantage over proteolytic digestion protocols because the chemical recognition sites result in the reproducible and consistent production of uniform molecular weight polypeptide standards due to the precise cleavage.

In addition, the proteases used in production of sets of molecular weight polypeptide standards may be difficult to fully remove, and the resultant standards may exhibit poor storage stability and/or poor crystallization when mixed with the matrix. Such problems are often exacerbated by the hostile conditions provided by the matrix compositions and solvents used in MALDI-TOF. On the other hand, the disclosed molecular weight polypeptide standards and methods of producing such standards provide storage stable standards.

Thus, the disclosed molecular weight polypeptide standards and methods of producing such standards maintain the benefits provided by current chemical synthesis and proteolytic digestion protocols while eliminating the disadvantages associated with both. Besides providing advantages over such current protocols, the disclosed molecular weight polypeptide standards and methods of producing such standards provide additional benefits. For example, the polypeptides disclosed herein are suitable for both negative and positive mode MALDI-TOF. In addition, the polypeptides disclosed herein require no incubation or other steps prior to analysis and as such are ready for use, are less expensive to manufacture, and are more environmentally friendly than current molecular weight polypeptide standards.

### Recombinant Protein

The present specification provides a recombinant protein comprising a plurality of repeating units each repeating unit including a desired molecular weight polypeptide standard and a recognition site of a site-specific chemical cleavage agent. A recombinant protein disclosed herein can optionally comprise a N- and/or a C-terminal purification or solubilization tag.

Is some embodiments, a recombinant protein comprising one or more repeating units, each of the one or more repeating units comprising a chemical cleavage site and a molecular weight polypeptide standard, wherein the chemical cleavage site is an amino acid sequence, the recombinant protein arranged according to formula I

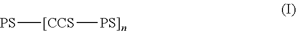

wherein brackets indicate a repeating unit of the one or more repeating units; PP is a molecular weight polypeptide standard; CCS is a chemical cleavage recognition site; and n is an integer from 1 to 50.

In some embodiments, a recombinant protein disclosed herein may further, and optionally, include a purification tag or a solubilization tag, the recombinant protein arranged according to formula II, III, IV, VI:

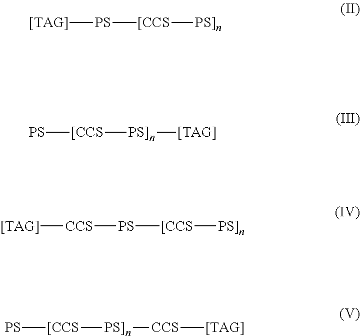

wherein brackets indicate a repeating unit of the one or more repeating units; PP is a molecular weight polypeptide standard; CCS is a chemical cleavage recognition site; TAG is the affinity purification tag or the solubilization tag; and n is an integer from 1 to 50.

The amino acid sequence of PP and CCS of a recombinant protein disclosed herein typically comprise any combination of the following amino acids: S, H, G, V, P, R, oxidized M (oxM), A, T, Q, D, E, P, L, and F. Amnio acids are chosen in a way to produce stable peptide sequences. In some embodiments the amino acid sequence of PP and CCS may also include I and/or L. In some embodiments, M and C are excluded.

In some embodiments methionine residues are oxidized prior to cleavage of the recombinant protein. This can be achieved by purging the reaction mixture comprising the recombinant protein with air in order to saturate the solution with oxygen. This avoids the gradual oxidation of methionine residues during storage which would result in a change in mass of the molecular weight polypeptide standards during storage. Oxidized methionine is stable thus maintains the mass. However, methionine is gradually converted to its oxidized form causing mass change for the calibration peptide. Following the hydrolysis, the peptide mixtures were fractionated using HPLC equipped with a C18 column.

### Molecular Weight Polypeptide Standards

The size of the recombinant protein and constituent molecular weight polypeptide standards is determined primarily by the molecular weights of polypeptide standards desired and the known size constraints associated with a chose expression system, such as, e.g., e.g. limitations on vector insert size and/or overall vector plus insert size in a particular host cell. General principles governing the use of recombinant expression systems and the selection of appropriate molecular weight standards for calibration of mass spectrometers are known in the art.

In some embodiments the repeating units comprise different molecular weight polypeptide standards that have different molecular weights due to differing amino acid lengths. Each different molecular weight polypeptide standard having a different molecular weight can be present in one or more repeating units of a recombinant protein disclosed herein. For example, a first molecular weight polypeptide standard will have a first molecular weight, a second molecular weight polypeptide standard will have a second molecular weight, a third molecular weight polypeptide standard will have a third molecular weight, a fourth molecular weight polypeptide standard will have a fourth molecular weight, a fifth molecular weight polypeptide standard will have a fifth molecular weight, a sixth molecular weight polypeptide standard will have a sixth molecular weight, a seventh molecular weight polypeptide standard will have a seventh molecular weight, an eighth molecular weight polypeptide standard will have a eighth molecular weight, a ninth molecular weight polypeptide standard will have a ninth molecular weight, and so on, with the first, second, third, fourth, fifth, sixth, seventh, eighth and ninth molecular weights each being a different molecular weight that the others. Accordingly, the recombinant protein as a whole comprises several different polypeptide standards each with a different molecular weight, with each polypeptide standard being present in one or more copies.

In some embodiments, a recombinant protein disclosed herein comprises at least one repeating unit comprising a molecular weight polypeptide standard having a molecular weight of about 0.5 kDa to about 4 kDa. In aspects of these embodiments, a recombinant protein disclosed herein comprises one repeating unit comprising a molecular weight polypeptide standard having a molecular weight of about 0.5 kDa to about 2 kDa, or one repeating unit comprising a molecular weight polypeptide standard having a molecular weight of about 2 kDa to about 4 kDa, or both. In other aspects of these embodiments, a recombinant protein disclosed herein comprises one repeating unit comprising a molecular weight polypeptide standard having a molecular weight of about 0.5 kDa to about 1 kDa, one repeating unit comprising a molecular weight polypeptide standard having a molecular weight of about 1 kDa to about 1.5 kDa, one repeating unit comprising a molecular weight polypeptide standard having a molecular weight of about 1.5 kDa to about 2 kDa, one repeating unit comprising a molecular weight polypeptide standard having a molecular weight of about 2 kDa to about 2.5 kDa, one repeating unit comprising a molecular weight polypeptide standard having a molecular weight of about 2.5 kDa to about 3 kDa, one repeating unit comprising a molecular weight polypeptide standard having a molecular weight of about 3 kDa to about 3.5 kDa, one repeating unit comprising a molecular weight polypeptide standard having a molecular weight of about 3.5 kDa to about 4 kDa, or any combination thereof.

In some embodiments, a recombinant protein disclosed herein comprises at least one repeating unit comprising a molecular weight polypeptide standard having a molecular weight of about 4 kDa to about 8 kDa. In aspects of these embodiments, a recombinant protein disclosed herein comprises one repeating unit comprising a molecular weight polypeptide standard having a molecular weight of about 4 kDa to about 6 kDa, or one repeating unit comprising a molecular weight polypeptide standard having a molecular weight of about 6 kDa to about 8 kDa, or both. In other aspects of these embodiments, a recombinant protein disclosed herein comprises one repeating unit comprising a molecular weight polypeptide standard having a molecular weight of about 4 kDa to about 4.5 kDa, one repeating unit comprising a molecular weight polypeptide standard having a molecular weight of about 4.5 kDa to about 5 kDa, one repeating unit comprising a molecular weight polypeptide standard having a molecular weight of about 5 kDa to about 5.5 kDa, one repeating unit comprising a molecular weight polypeptide standard having a molecular weight of about 5.5 kDa to about 6 kDa, one repeating unit comprising a molecular weight polypeptide standard having a molecular weight of about 6 kDa to about 6.5 kDa, one repeating unit comprising a molecular weight polypeptide standard having a molecular weight of about 6.5 kDa to about 7 kDa, one repeating unit comprising a molecular weight polypeptide standard having a molecular weight of about 7 kDa to about 7.5 kDa, one repeating unit comprising a molecular weight polypeptide standard having a molecular weight of about 7.5 kDa to about 8 kDa, or any combination thereof.

In some embodiments, a recombinant protein disclosed herein comprises at least one repeating unit comprising a molecular weight polypeptide standard having a molecular weight of about 8 kDa to about 12 kDa. In aspects of these embodiments, a recombinant protein disclosed herein comprises one repeating unit comprising a molecular weight polypeptide standard having a molecular weight of about 8 kDa to about 10 kDa, or one repeating unit comprising a molecular weight polypeptide standard having a molecular weight of about 10 kDa to about 12 kDa, or both. In other aspects of these embodiments, a recombinant protein disclosed herein comprises one repeating unit comprising a molecular weight polypeptide standard having a molecular weight of about 8 kDa to about 8.5 kDa, one repeating unit comprising a molecular weight polypeptide standard having a molecular weight of about 8.5 kDa to about 9 kDa, one repeating unit comprising a molecular weight polypeptide standard having a molecular weight of about 9 kDa to about 9.5 kDa, one repeating unit comprising a molecular weight polypeptide standard having a molecular weight of about 9.5 kDa to about 10 kDa, one repeating unit comprising a molecular weight polypeptide standard having a molecular weight of about 10 kDa to about 10.5 kDa, one repeating unit comprising a molecular weight polypeptide standard having a molecular weight of about 10.5 kDa to about 11 kDa, one repeating unit comprising a molecular weight polypeptide standard having a molecular weight of about 11 kDa to about 11.5 kDa, or one repeating unit comprising a molecular weight polypeptide standard having a molecular weight of about 11.5 kDa to about 12 kDa, or any combination thereof.

In some embodiments, a recombinant protein disclosed herein comprises at least one repeating unit comprising a molecular weight polypeptide standard having a molecular weight of about 12 kDa to about 16 kDa. In aspects of these embodiments, a recombinant protein disclosed herein comprises one repeating unit comprising a molecular weight polypeptide standard having a molecular weight of about 12 kDa to about 14 kDa, or one repeating unit comprising a molecular weight polypeptide standard having a molecular weight of about 14 kDa to about 16 kDa, or both. In other aspects of these embodiments, a recombinant protein disclosed herein comprises one repeating unit comprising a molecular weight polypeptide standard having a molecular weight of about 12 kDa to about 12.5 kDa, one repeating unit comprising a molecular weight polypeptide standard having a molecular weight of about 12.5 kDa to about 13 kDa, one repeating unit comprising a molecular weight polypeptide standard having a molecular weight of about 13 kDa to about 13.5 kDa, one repeating unit comprising a molecular weight polypeptide standard having a molecular weight of about 13.5 kDa to about 14 kDa, one repeating unit comprising a molecular weight polypeptide standard having a molecular weight of about 14 kDa to about 14.5 kDa, one repeating unit comprising a molecular weight polypeptide standard having a molecular weight of about 14.5 kDa to about 15 kDa, one repeating unit comprising a molecular weight polypeptide standard having a molecular weight of about 15 kDa to about 15.5 kDa, one repeating unit comprising a molecular weight polypeptide standard having a molecular weight of about 15.5 kDa to about 16 kDa, or any combination thereof.

In some embodiments, a recombinant protein disclosed herein comprises at least one repeating unit comprising a molecular weight polypeptide standard having a molecular weight of about 16 kDa to about 20 kDa. In aspects of these embodiments, a recombinant protein disclosed herein comprises one repeating unit comprising a molecular weight polypeptide standard having a molecular weight of about 16 kDa to about 18 kDa, or one repeating unit comprises a molecular weight polypeptide standard having a molecular weight of about 18 kDa to about 20 kDa, or both. In other aspects of these embodiments, a recombinant protein disclosed herein comprises one repeating unit comprising a molecular weight polypeptide standard having a molecular weight of about 16 kDa to about 16.5 kDa, one repeating unit comprising a molecular weight polypeptide standard having a molecular weight of about 16.5 kDa to about 17 kDa, one repeating unit comprising a molecular weight polypeptide standard having a molecular weight of about 17 kDa to about 17.5 kDa, one repeating unit comprising a molecular weight polypeptide standard having a molecular weight of about 17.5 kDa to about 18 kDa, one repeating unit comprising a molecular weight polypeptide standard having a molecular weight of about 18 kDa to about 18.5 kDa, one repeating unit comprising a molecular weight polypeptide standard having a molecular weight of about 18.5 kDa to about 19 kDa, one repeating unit comprising a molecular weight polypeptide standard having a molecular weight of about 19 kDa to about 19.5 kDa, one repeating unit comprising a molecular weight polypeptide standard having a molecular weight of about 19.5 kDa to about 20 kDa, or any combination thereof.

In some embodiments, a recombinant protein disclosed herein comprises at least one repeating unit comprising a molecular weight polypeptide standard having a molecular weight of about 20 kDa to about 24 kDa. In aspects of these embodiments, a recombinant protein disclosed herein comprises one repeating unit comprising a molecular weight polypeptide standard having a molecular weight of about 20 kDa to about 22 kDa, or one repeating unit comprising a molecular weight polypeptide standard having a molecular weight of about 22 kDa to about 24 kDa, or both. In other aspects of these embodiments, a recombinant protein disclosed herein comprises one repeating unit comprising a molecular weight polypeptide standard having a molecular weight of about 20 kDa to about 20.5 kDa, one repeating unit comprising a molecular weight polypeptide standard having a molecular weight of about 20.5 kDa to about 21 kDa, one repeating unit comprising a molecular weight polypeptide standard having a molecular weight of about 21 kDa to about 21.5 kDa, one repeating unit comprising a molecular weight polypeptide standard having a molecular weight of about 21.5 kDa to about 22 kDa, one repeating unit comprising a molecular weight polypeptide standard having a molecular weight of about 22 kDa to about 22.5 kDa, one repeating unit comprising a molecular weight polypeptide standard having a molecular weight of about 22.5 kDa to about 23 kDa, one repeating unit comprising a molecular weight polypeptide standard having a molecular weight of about 23 kDa to about 23.5 kDa, one repeating unit comprising a molecular weight polypeptide standard having a molecular weight of about 23.5 kDa to about 24 kDa, or any combination thereof.

In some embodiments, a recombinant protein disclosed herein comprises at least one repeating unit comprising a molecular weight polypeptide standard having a molecular weight of about 24 kDa to about 28 kDa. In aspects of these embodiments, a recombinant protein disclosed herein comprises one repeating unit comprising a molecular weight polypeptide standard having a molecular weight of about 24 kDa to about 26 kDa, or one repeating unit comprising a molecular weight polypeptide standard having a molecular weight of about 26 kDa to about 28 kDa, or both. In other aspects of these embodiments, a recombinant protein disclosed herein comprises one repeating unit comprising a molecular weight polypeptide standard having a molecular weight of about 24 kDa to about 24.5 kDa, one repeating unit comprising a molecular weight polypeptide standard having a molecular weight of about 24.5 kDa to about 25 kDa, one repeating unit comprising a molecular weight polypeptide standard having a molecular weight of about 25 kDa to about 25.5 kDa, one repeating unit comprising a molecular weight polypeptide standard having a molecular weight of about 25.5 kDa to about 26 kDa, one repeating unit comprising a molecular weight polypeptide standard having a molecular weight of about 26 kDa to about 26.5 kDa, one repeating unit comprising a molecular weight polypeptide standard having a molecular weight of about 26.5 kDa to about 27 kDa, one repeating unit comprising a molecular weight polypeptide standard having a molecular weight of about 27 kDa to about 27.5 kDa, one repeating unit comprising a molecular weight polypeptide standard having a molecular weight of about 27.5 kDa to about 28 kDa, or any combination thereof.

In some embodiments, a recombinant protein disclosed herein comprises at least one repeating unit comprising a molecular weight polypeptide standard having a molecular weight of about 28 kDa to about 32 kDa. In aspects of these embodiments, a recombinant protein disclosed herein comprises one repeating unit comprising a molecular weight polypeptide standard having a molecular weight of about 28 kDa to about 30 kDa, or one repeating unit comprising a molecular weight polypeptide standard having a molecular weight of about 30 kDa to about 32 kDa, or both. In other aspects of these embodiments, a recombinant protein disclosed herein comprises one repeating unit comprising a molecular weight polypeptide standard having a molecular weight of about 28 kDa to about 28.5 kDa, one repeating unit comprising a molecular weight polypeptide standard having a molecular weight of about 28.5 kDa to about 29 kDa, one repeating unit comprising a molecular weight polypeptide standard having a molecular weight of about 29 kDa to about 29.5 kDa, one repeating unit comprising a molecular weight polypeptide standard having a molecular weight of about 29.5 kDa to about 30 kDa, one repeating unit comprising a molecular weight polypeptide standard having a molecular weight of about 30 kDa to about 30.5 kDa, one repeating unit comprising a molecular weight polypeptide standard having a molecular weight of about 30.5 kDa to about 31 kDa, one repeating unit comprising a molecular weight polypeptide standard having a molecular weight of about 31 kDa to about 31.5 kDa, one repeating unit comprising a molecular weight polypeptide standard having a molecular weight of about 31.5 kDa to about 32 kDa, or any combination thereof.

In some embodiments, a recombinant protein disclosed herein comprises at least one repeating unit comprising a molecular weight polypeptide standard having a molecular weight of about 32 kDa to about 36 kDa. In aspects of these embodiments, a recombinant protein disclosed herein comprises one repeating unit comprising a molecular weight polypeptide standard having a molecular weight of about 32 kDa to about 34 kDa, or one repeating unit comprising a molecular weight polypeptide standard having a molecular weight of about 34 kDa to about 36 kDa, or both. In other aspects of these embodiments, a recombinant protein disclosed herein comprises one repeating unit comprising a molecular weight polypeptide standard having a molecular weight of about 32 kDa to about 32.5 kDa, one repeating unit comprising a molecular weight polypeptide standard having a molecular weight of about 32.5 kDa to about 33 kDa, one repeating unit comprising a molecular weight polypeptide standard having a molecular weight of about 33 kDa to about 33.5 kDa, one repeating unit comprising a molecular weight polypeptide standard having a molecular weight of about 33.5 kDa to about 34 kDa, one repeating unit comprising a molecular weight polypeptide standard having a molecular weight of about 34 kDa to about 34.5 kDa, one repeating unit comprising a molecular weight polypeptide standard having a molecular weight of about 34.5 kDa to about 35 kDa, one repeating unit comprising a molecular weight polypeptide standard having a molecular weight of about 35 kDa to about 35.5 kDa, one repeating unit comprising a molecular weight polypeptide standard having a molecular weight of about 35.5 kDa to about 36 kDa.

In some embodiments, a recombinant protein disclosed herein comprises at least one repeating unit comprising a molecular weight polypeptide standard having a molecular weight of about 36 kDa to about 40 kDa. In aspects of these embodiments, a recombinant protein disclosed herein comprises one repeating unit comprising a molecular weight polypeptide standard having a molecular weight of about 36 kDa to about 38 kDa, or one repeating unit comprising a molecular weight polypeptide standard having a molecular weight of about 38 kDa to about 40 kDa, or both. In other aspects of these embodiments, a recombinant protein disclosed herein comprises one repeating unit comprising a molecular weight polypeptide standard having a molecular weight of about 36 kDa to about 36.5 kDa, one repeating unit comprising a molecular weight polypeptide standard having a molecular weight of about 36.5 kDa to about 37 kDa, one repeating unit comprising a molecular weight polypeptide standard having a molecular weight of about 37 kDa to about 37.5 kDa, one repeating unit comprising a molecular weight polypeptide standard having a molecular weight of about 37.5 kDa to about 38 kDa, one repeating unit comprising a molecular weight polypeptide standard having a molecular weight of about 38 kDa to about 38.5 kDa, one repeating unit comprising a molecular weight polypeptide standard having a molecular weight of about 38.5 kDa to about 39 kDa, one repeating unit comprising a molecular weight polypeptide standard having a molecular weight of about 39 kDa to about 39.5 kDa, one repeating unit comprising a molecular weight polypeptide standard having a molecular weight of about 39.5 kDa to about 40 kDa, or any combination thereof.

In some embodiments, a recombinant protein disclosed herein comprises at least one repeating unit comprising a molecular weight polypeptide standard having a molecular weight of about 40 kDa to about 44 kDa. In aspects of these embodiments, a recombinant protein disclosed herein comprises one repeating unit comprising a molecular weight polypeptide standard having a molecular weight of about 40 kDa to about 42 kDa, or one repeating unit comprising a molecular weight polypeptide standard having a molecular weight of about 42 kDa to about 44 kDa, or both. In other aspects of these embodiments, a recombinant protein disclosed herein comprises one repeating unit comprising a molecular weight polypeptide standard having a molecular weight of about 40 kDa to about 40.5 kDa, one repeating unit comprising a molecular weight polypeptide standard having a molecular weight of about 40.5 kDa to about 41 kDa, one repeating unit comprising a molecular weight polypeptide standard having a molecular weight of about 41 kDa to about 41.5 kDa, one repeating unit comprising a molecular weight polypeptide standard having a molecular weight of about 41.5 kDa to about 42 kDa, one repeating unit comprising a molecular weight polypeptide standard having a molecular weight of about 42 kDa to about 42.5 kDa, one repeating unit comprising a molecular weight polypeptide standard having a molecular weight of about 42.5 kDa to about 43 kDa, one repeating unit comprising a molecular weight polypeptide standard having a molecular weight of about 43 kDa to about 43.5 kDa, one repeating unit comprising a molecular weight polypeptide standard having a molecular weight of about 43.5 kDa to about 44 kDa, or any combination thereof.

In some embodiments, a recombinant protein disclosed herein comprises at least one repeating unit comprising a molecular weight polypeptide standard having a molecular weight of about 44 kDa to about 48 kDa. In aspects of these embodiments, a recombinant protein disclosed herein comprises one repeating unit comprising a molecular weight polypeptide standard having a molecular weight of about 44 kDa to about 46 kDa, or one repeating unit comprising a molecular weight polypeptide standard having a molecular weight of about 46 kDa to about 48 kDa, or both. In other aspects of these embodiments, a recombinant protein disclosed herein comprises one repeating unit comprising a molecular weight polypeptide standard having a molecular weight of about 44 kDa to about 44.5 kDa, one repeating unit comprising a molecular weight polypeptide standard having a molecular weight of about 44.5 kDa to about 45 kDa, one repeating unit comprising a molecular weight polypeptide standard having a molecular weight of about 45 kDa to about 45.5 kDa, one repeating unit comprising a molecular weight polypeptide standard having a molecular weight of about 45.5 kDa to about 46 kDa, one repeating unit comprising a molecular weight polypeptide standard having a molecular weight of about 46 kDa to about 46.5 kDa, one repeating unit comprising a molecular weight polypeptide standard having a molecular weight of about 46.5 kDa to about 47 kDa, one repeating unit comprising a molecular weight polypeptide standard having a molecular weight of about 47 kDa to about 47.5 kDa, one repeating unit comprising a molecular weight polypeptide standard having a molecular weight of about 47.5 kDa to about 48 kDa, or any combination thereof.

In some embodiments, a recombinant protein disclosed herein comprises at least one repeating unit comprising a molecular weight polypeptide standard having a molecular weight of about 48 kDa to about 52 kDa. In aspects of these embodiments, a recombinant protein disclosed herein comprises one repeating unit comprising a molecular weight polypeptide standard having a molecular weight of about 48 kDa to about 50 kDa, or one repeating unit comprising a molecular weight polypeptide standard having a molecular weight of about 50 kDa to about 52 kDa, or both. In other aspects of these embodiments, a recombinant protein disclosed herein comprises one repeating unit comprising a molecular weight polypeptide standard having a molecular weight of about 48 kDa to about 48.5 kDa, one repeating unit comprising a molecular weight polypeptide standard having a molecular weight of about 48.5 kDa to about 49 kDa, one repeating unit comprising a molecular weight polypeptide standard having a molecular weight of about 49 kDa to about 49.5 kDa, one repeating unit comprising a molecular weight polypeptide standard having a molecular weight of about 49.5 kDa to about 50 kDa, one repeating unit comprising a molecular weight polypeptide standard having a molecular weight of about 50 kDa to about 50.5 kDa, one repeating unit comprising a molecular weight polypeptide standard having a molecular weight of about 50.5 kDa to about 51 kDa, one repeating unit comprising a molecular weight polypeptide standard having a molecular weight of about 51 kDa to about 51.5 kDa, one repeating unit comprising a molecular weight polypeptide standard having a molecular weight of about 51.5 kDa to about 52 kDa, or any combination thereof.

A plurality of repeating units disclosed herein can comprise degenerate molecular weight polypeptide standards. Degenerate molecular weight polypeptide standards disclosed herein are polypeptide standards that have the same molecular weight but differ in their amino acid sequence. An advantage of including such degenerate sequences is that each peptide has its own physicochemical properties while having the exact same mass. For example, Yang et al., 2007, Journal of Proteome Research 7, pages 62-69 revealed that the locations and types of amino acids in a peptide have a strong relationship to peak intensity for peptide analysis by MALDI-TOF mass spectrometry. One reason is that the primary sequence of a peptide affects its basicity in the gas phase as shown in Table 1 for the following three pentapeptides as an example, in which B_{(g)}+H⁺_{(g)}→BH⁺_{(g)}, −ΔGo=GB=Gas phase basicity.

TABLE 1
Pentapeptide Example

| Peptide | GB (kcal/mol) | SEQ ID NO: |
|---|---|---|
| GlyLysLysGlyGly | 237.8 | 97 |
| GlyLysGlyLysGly | 238.5 | 98 |
| LysGlyGlyGlyLys | 241.9 | 99 |

Another advantage of including degenerate molecular weight polypeptide standards is that their presence eliminates or at least significantly reduces the likelihood of analyte suppression (Richard Knochenmuss, New Developments in Mass Spectrometry No. 12, Chapter 1, pages 3-19). For example, some analytes can completely suppress others through the following mechanism:

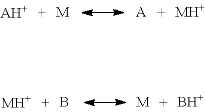

giving a net reaction:

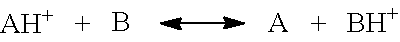

in which M is the matrix, A and B are the analytes.

Therefore, the choice of polypeptide standards and their ratio is preferably such that the presence of one polypeptide standard should not completely suppress the other ions. The use of degenerate molecular weight polypeptide standards diversifies the polypeptides in the standard to increase the chance of polypeptides being seen in the spectrum which would also translate into having a wider mass coverage.

A further advantage of including degenerate molecular weight polypeptide standards is that their presence improves reproducibility. A problem with MALDI-TOF is poor reproducibility (sample-to sample and one laser shot to another) which appears to be a consequence of poor crystallization (Israr et al., Clin Chem Lab Med 2020, 58 (6) pages 883-896). The crystallization is affected by both analyte and the matrix. The use of degenerate molecular weight polypeptide standards increases the chance of forming suitable crystals that provide better ionization and thus more intense signals. In addition, the use of degenerate molecular weight polypeptide standards can also reduce the instrument dependent variability that is frequently observed in MALDI-TOF instruments.

Accordingly, the use of degenerate molecular weight polypeptide standards can increase the chance of ionization without experimental examination of each polypeptide standard. This increased possibility of the presence of at least one polypeptide standard that can be properly ionized and excited to generate a peak.

In some embodiments the repeating units comprise a plurality of degenerate molecular weight polypeptide standards that have the same molecular weight but differ in their amino acid sequence. Each degenerate molecular weight polypeptide standard having the same molecular weight can be present in one or more repeating units of a recombinant protein disclosed herein. In aspects of these embodiments, two or more degenerate molecular weight polypeptide standards can have the same combination of amino acids but in a different order. In other aspects of these embodiments, two or more degenerate molecular weight polypeptide standards can have the same molecular weight but a different amino acid sequence due to amino acid substitutions that individually are of the same molecular weight (e.g. leucine and isoleucine) or combinations of amino acid substitutions that in sum have the same molecular weight. For example, a first degenerative molecular weight polypeptide standard will have the molecular weight, as a second, third, fourth, or fifth degenerative molecular weight polypeptide standards even though each polypeptide standard has the can have the same or similar amino acids content. Accordingly, the recombinant protein as a whole comprises several degenerate polypeptide standards of the same molecular weight, with each polypeptide standard being present in one or more copies.

In aspects of these embodiments, a plurality of degenerate molecular weight polypeptide standards each having the same molecular weight and the same or similar combination of amino acids but differing in the order of the amino acids in their amino acid sequence can be, e.g., two, three, four, five, six, or seven degenerate molecular weight polypeptide standards, with each degenerate molecular weight polypeptide standard having the same molecular weight but differ in their amino acid sequence. In other aspects of these embodiments, a plurality of degenerate molecular weight polypeptide standards each having the same molecular weight and same or similar combination of amino acids but differing in the order of the amino acids in their amino acid sequence can be, e.g., at least two, at least three, at least four, at least five, at least six, or at least seven degenerate molecular weight polypeptide standards, with each degenerate molecular weight polypeptide standard having the same molecular weight but differ in their amino acid sequence. In yet other aspects of these embodiments, a plurality of degenerate molecular weight polypeptide standards each having the same molecular weight and same or similar combination of amino acids but differing in the order of the amino acids in their amino acid sequence can be, e.g., at most two, at most three, at most four, at most five, at most six, or at most seven degenerate molecular weight polypeptide standards, with each degenerate molecular weight polypeptide standard having the same molecular weight but differ in their amino acid sequence. In still other aspects of these embodiments, a plurality of degenerate molecular weight polypeptide standards each having the same molecular weight and same or similar combination of amino acids but differing in the order of the amino acids in their amino acid sequence can be, e.g., two to three, two to four, two to five, two to six, two to seven, three to four, three to five, three to six, three to seven, four to five, four to six, four to seven, five to six, five to seven, or six to seven, degenerate molecular weight polypeptide standards, with each degenerate molecular weight polypeptide standard having the same molecular weight but differ in their amino acid sequence.

In some embodiments, a recombinant protein disclosed herein comprising a plurality of repeating units can comprise a plurality of degenerate molecular weight polypeptide standards each of the plurality of degenerate molecular weight polypeptide standards having a different molecular weight. In aspects of these embodiments, a recombinant protein disclosed herein can comprise, e.g., two, three, four, five, six, or seven repeating units with each repeating comprising a degenerate molecular weight polypeptide standard having a different molecular weight. In other aspects of these embodiments, a recombinant protein disclosed herein can comprise, e.g., at least two, at least three, at least four, at least five, at least six, or at least seven repeating units with each repeating comprising a degenerate molecular weight polypeptide standard having a different molecular weight. In yet other aspects of these embodiments, a recombinant protein disclosed herein can comprise, e.g., at most two, at most three, at most four, at most five, at most six, or at most seven repeating units with each repeating comprising a degenerate molecular weight polypeptide standard having a different molecular weight. In still other aspects of these embodiments, a recombinant protein disclosed herein can comprise, e.g., two to three, two to four, two to five, two to six, two to seven, three to four, three to five, three to six, three to seven, four to five, four to six, four to seven, five to six, five to seven, or six to seven repeating units, with each repeating comprising a degenerate molecular weight polypeptide standard having a different molecular weight.

In some embodiments, a recombinant protein disclosed herein comprises at least two repeating units, with each repeating unit comprising a degenerate molecular weight polypeptide standard having the same molecular weight of about 0.5 kDa to about 4 kDa. In aspects of these embodiments, a recombinant protein disclosed herein comprises at least two repeating units, with each repeating unit comprising a degenerate molecular weight polypeptide standard having the same molecular weight of about 0.5 kDa to about 2 kDa, or with each repeating unit comprising a degenerate molecular weight polypeptide standard having the same molecular weight of about 2 kDa to about 4 kDa. In other aspects of these embodiments, a recombinant protein disclosed herein comprises at least two repeating units, with each repeating unit comprising a degenerate molecular weight polypeptide standard having the same molecular weight of about 0.5 kDa to about 1 kDa, the same molecular weight of about 1 kDa to about 1.5 kDa, the same molecular weight of about 1.5 kDa to about 2 kDa, the same molecular weight of about 2 kDa to about 2.5 kDa, the same molecular weight of about 2.5 kDa to about 3 kDa, the same molecular weight of about 3 kDa to about 3.5 kDa, or the same molecular weight of about 3.5 kDa to about 4 kDa.

In some embodiments, a recombinant protein disclosed herein comprises at least two repeating units, with each repeating unit comprising a degenerate molecular weight polypeptide standard having the same molecular weight of about 4 kDa to about 8 kDa. In aspects of these embodiments, a recombinant protein disclosed herein comprises at least two repeating units, with each repeating unit comprising a degenerate molecular weight polypeptide standard having the same molecular weight of about 4 kDa to about 6 kDa, or with each repeating unit comprising a degenerate molecular weight polypeptide standard having the same molecular weight of about 6 kDa to about 8 kDa. In other aspects of these embodiments, a recombinant protein disclosed herein comprises at least two repeating units, with each repeating unit comprising a degenerate molecular weight polypeptide standard having the same molecular weight of about 4 kDa to about 4.5 kDa, the same molecular weight of about 4.5 kDa to about 5 kDa, the same molecular weight of about 5 kDa to about 5.5 kDa, the same molecular weight of about 5.5 kDa to about 6 kDa, the same molecular weight of about 6 kDa to about 6.5 kDa, the same molecular weight of about 6.5 kDa to about 7 kDa, the same molecular weight of about 7 kDa to about 7.5 kDa, or the same molecular weight of about 7.5 kDa to about 8 kDa.

In some embodiments, a recombinant protein disclosed herein comprises at least two repeating units, with each repeating unit comprising a degenerate molecular weight polypeptide standard having the same molecular weight of about 8 kDa to about 12 kDa. In aspects of these embodiments, a recombinant protein disclosed herein comprises at least two repeating units, with each repeating unit comprising a degenerate molecular weight polypeptide standard having the same molecular weight of about 8 kDa to about 10 kDa, or or with each repeating unit comprising a degenerate molecular weight polypeptide standard having the same molecular weight of about 10 kDa to about 12 kDa. In other aspects of these embodiments, a recombinant protein disclosed herein comprises at least two repeating units, with each repeating unit comprising a degenerate molecular weight polypeptide standard having the same molecular weight of about 8 kDa to about 8.5 kDa, the same molecular weight of about 8.5 kDa to about 9 kDa, the same molecular weight of about 9 kDa to about 9.5 kDa, the same molecular weight of about 9.5 kDa to about 10 kDa, the same molecular weight of about 10 kDa to about 10.5 kDa, the same molecular weight of about 10.5 kDa to about 11 kDa, the same molecular weight of about 11 kDa to about 11.5 kDa, or the same molecular weight of about 11.5 kDa to about 12 kDa.

In some embodiments, a recombinant protein disclosed herein comprises at least two repeating units, with each repeating unit comprising a degenerate molecular weight polypeptide standard having the same molecular weight of about 12 kDa to about 16 kDa. In aspects of these embodiments, a recombinant protein disclosed herein comprises at least two repeating units, with each repeating unit comprising a degenerate molecular weight polypeptide standard having the same molecular weight of about 12 kDa to about 14 kDa, or with each repeating unit comprising a degenerate molecular weight polypeptide standard having the same molecular weight of about 14 kDa to about 16 kDa. In other aspects of these embodiments, a recombinant protein disclosed herein comprises at least two repeating units, with each repeating unit comprising a degenerate molecular weight polypeptide standard having the same molecular weight of about 12 kDa to about 12.5 kDa, the same molecular weight of about 12.5 kDa to about 13 kDa, the same molecular weight of about 13 kDa to about 13.5 kDa, the same molecular weight of about 13.5 kDa to about 14 kDa, the same molecular weight of about 14 kDa to about 14.5 kDa, the same molecular weight of about 14.5 kDa to about 15 kDa, the same molecular weight of about 15 kDa to about 15.5 kDa, or the same molecular weight of about 15.5 kDa to about 16 kDa.

In some embodiments, a recombinant protein disclosed herein comprises at least two repeating units, with each repeating unit comprising a degenerate molecular weight polypeptide standard having the same molecular weight of about 16 kDa to about 20 kDa. In aspects of these embodiments, a recombinant protein disclosed herein comprises at least two repeating units, with each repeating unit comprising a degenerate molecular weight polypeptide standard having the same molecular weight of about 16 kDa to about 18 kDa, or with each repeating unit comprising a degenerate molecular weight polypeptide standard having the same molecular weight of about 18 kDa to about 20 kDa. In other aspects of these embodiments, a recombinant protein disclosed herein comprises at least two repeating units, with each repeating unit comprising a degenerate molecular weight polypeptide standard having the same molecular weight of about 16 kDa to about 16.5 kDa, the same molecular weight of about 16.5 kDa to about 17 kDa, the same molecular weight of about 17 kDa to about 17.5 kDa, the same molecular weight of about 17.5 kDa to about 18 kDa, the same molecular weight of about 18 kDa to about 18.5 kDa, the same molecular weight of about 18.5 kDa to about 19 kDa, the same molecular weight of about 19 kDa to about 19.5 kDa, or the same molecular weight of about 19.5 kDa to about 20 kDa.

In some embodiments, a recombinant protein disclosed herein comprises at least two repeating units, with each repeating unit comprising a degenerate molecular weight polypeptide standard having the same molecular weight of about 20 kDa to about 24 kDa. In aspects of these embodiments, a recombinant protein disclosed herein comprises at least two repeating units, with each repeating unit comprising a degenerate molecular weight polypeptide standard having the same molecular weight of about 20 kDa to about 22 kDa, or with each repeating unit comprising a degenerate molecular weight polypeptide standard having the same molecular weight of about 22 kDa to about 24 kDa. In other aspects of these embodiments, a recombinant protein disclosed herein comprises at least two repeating units, with each repeating unit comprising a degenerate molecular weight polypeptide standard having the same molecular weight of about 20 kDa to about 20.5 kDa, the same molecular weight of about 20.5 kDa to about 21 kDa, the same molecular weight of about 21 kDa to about 21.5 kDa, the same molecular weight of about 21.5 kDa to about 22 kDa, the same molecular weight of about 22 kDa to about 22.5 kDa, the same molecular weight of about 22.5 kDa to about 23 kDa, the same molecular weight of about 23 kDa to about 23.5 kDa, or the same molecular weight of about 23.5 kDa to about 24 kDa.

In some embodiments, a recombinant protein disclosed herein comprises at least two repeating units, with each repeating unit comprising a degenerate molecular weight polypeptide standard having the same molecular weight of about 24 kDa to about 28 kDa. In aspects of these embodiments, a recombinant protein disclosed herein comprises at least two repeating units, with each repeating unit comprising a degenerate molecular weight polypeptide standard having the same molecular weight of about 24 kDa to about 26 kDa, or with each repeating unit comprising a degenerate molecular weight polypeptide standard having the same molecular weight of about 26 kDa to about 28 kDa. In other aspects of these embodiments, a recombinant protein disclosed herein comprises at least two repeating units, with each repeating unit comprising a degenerate molecular weight polypeptide standard having the same molecular weight of about 24 kDa to about 24.5 kDa, the same molecular weight of about 24.5 kDa to about 25 kDa, the same molecular weight of about 25 kDa to about 25.5 kDa, the same molecular weight of about 25.5 kDa to about 26 kDa, the same molecular weight of about 26 kDa to about 26.5 kDa, the same molecular weight of about 26.5 kDa to about 27 kDa, the same molecular weight of about 27 kDa to about 27.5 kDa, or the same molecular weight of about 27.5 kDa to about 28 kDa.

In some embodiments, a recombinant protein disclosed herein comprises at least two repeating units, with each repeating unit comprising a degenerate molecular weight polypeptide standard having the same molecular weight of about 28 kDa to about 32 kDa. In aspects of these embodiments, a recombinant protein disclosed herein comprises at least two repeating units, with each repeating unit comprising a degenerate molecular weight polypeptide standard having the same molecular weight of about 28 kDa to about 30 kDa, or with each repeating unit comprising a degenerate molecular weight polypeptide standard having the same molecular weight of about 30 kDa to about 32 kDa. In other aspects of these embodiments, a recombinant protein disclosed herein comprises at least two repeating units, with each repeating unit comprising a degenerate molecular weight polypeptide standard having the same molecular weight of about 28 kDa to about 28.5 kDa, the same molecular weight of about 28.5 kDa to about 29 kDa, the same molecular weight of about 29 kDa to about 29.5 kDa, the same molecular weight of about 29.5 kDa to about 30 kDa, the same molecular weight of about 30 kDa to about 30.5 kDa, the same molecular weight of about 30.5 kDa to about 31 kDa, the same molecular weight of about 31 kDa to about 31.5 kDa, or the same molecular weight of about 31.5 kDa to about 32 kDa.

In some embodiments, a recombinant protein disclosed herein comprises at least two repeating units, with each repeating unit comprising a degenerate molecular weight polypeptide standard having the same molecular weight of about 32 kDa to about 36 kDa. In aspects of these embodiments a recombinant protein disclosed herein comprises at least two repeating units, with each repeating unit comprising a degenerate molecular weight polypeptide standard having the same molecular weight of about 32 kDa to about 34 kDa, or with each repeating unit comprising a degenerate molecular weight polypeptide standard having the same molecular weight of about 34 kDa to about 36 kDa. In other aspects of these embodiments, a recombinant protein disclosed herein comprises at least two repeating units, with each repeating unit comprising a degenerate molecular weight polypeptide standard having the same molecular weight of about 32 kDa to about 32.5 kDa, the same molecular weight of about 32.5 kDa to about 33 kDa, the same molecular weight of about 33 kDa to about 33.5 kDa, the same molecular weight of about 33.5 kDa to about 34 kDa, the same molecular weight of about 34 kDa to about 34.5 kDa, the same molecular weight of about 34.5 kDa to about 35 kDa, the same molecular weight of about 35 kDa to about 35.5 kDa, or the same molecular weight of about 35.5 kDa to about 36 kDa.

In some embodiments, a recombinant protein disclosed herein comprises at least two repeating units, with each repeating unit comprising a degenerate molecular weight polypeptide standard having the same molecular weight of about 36 kDa to about 40 kDa. In aspects of these embodiments, a recombinant protein disclosed herein comprises at least two repeating units, with each repeating unit comprising a degenerate molecular weight polypeptide standard having the same molecular weight of about 36 kDa to about 38 kDa, or with each repeating unit comprising a degenerate molecular weight polypeptide standard having the same molecular weight of about 38 kDa to about 40 kDa. In other aspects of these embodiments, a recombinant protein disclosed herein comprises at least two repeating units, with each repeating unit comprising a degenerate molecular weight polypeptide standard having the same molecular weight of about 36 kDa to about 36.5 kDa, the same molecular weight of about 36.5 kDa to about 37 kDa, the same molecular weight of about 37 kDa to about 37.5 kDa, the same molecular weight of about 37.5 kDa to about 38 kDa, the same molecular weight of about 38 kDa to about 38.5 kDa, the same molecular weight of about 38.5 kDa to about 39 kDa, the same molecular weight of about 39 kDa to about 39.5 kDa, or the same molecular weight of about 39.5 kDa to about 40 kDa.

In some embodiments, a recombinant protein disclosed herein comprises at least two repeating units, with each repeating unit comprising a degenerate molecular weight polypeptide standard having the same molecular weight of about 40 kDa to about 44 kDa. In aspects of these embodiments, a recombinant protein disclosed herein comprises at least two repeating units, with each repeating unit comprising a degenerate molecular weight polypeptide standard having the same molecular weight of about 40 kDa to about 42 kDa, or with each repeating unit comprising a degenerate molecular weight polypeptide standard having the same molecular weight of about 42 kDa to about 44 kDa. In other aspects of these embodiments, a recombinant protein disclosed herein comprises at least two repeating units, with each repeating unit comprising a degenerate molecular weight polypeptide standard having the same molecular weight of about 40 kDa to about 40.5 kDa, the same molecular weight of about 40.5 kDa to about 41 kDa, the same molecular weight of about 41 kDa to about 41.5 kDa, the same molecular weight of about 41.5 kDa to about 42 kDa, the same molecular weight of about 42 kDa to about 42.5 kDa, the same molecular weight of about 42.5 kDa to about 43 kDa, the same molecular weight of about 43 kDa to about 43.5 kDa, or the same molecular weight of about 43.5 kDa to about 44 kDa.

In some embodiments, a recombinant protein disclosed herein comprises at least two repeating units, with each repeating unit comprising a degenerate molecular weight polypeptide standard having the same molecular weight of about 44 kDa to about 48 kDa. In aspects of these embodiments, a recombinant protein disclosed herein comprises at least two repeating units, with each repeating unit comprising a degenerate molecular weight polypeptide standard having the same molecular weight of about 44 kDa to about 46 kDa, or with each repeating unit comprising a degenerate molecular weight polypeptide standard having the same molecular weight of about 46 kDa to about 48 kDa. In other aspects of these embodiments, a recombinant protein disclosed herein comprises at least two repeating units, with each repeating unit comprising a degenerate molecular weight polypeptide standard having the same molecular weight of about 44 kDa to about 44.5 kDa, the same molecular weight of about 44.5 kDa to about 45 kDa, the same molecular weight of about 45 kDa to about 45.5 kDa, the same molecular weight of about 45.5 kDa to about 46 kDa, the same molecular weight of about 46 kDa to about 46.5 kDa, the same molecular weight of about 46.5 kDa to about 47 kDa, the same molecular weight of about 47 kDa to about 47.5 kDa, or the same molecular weight of about 47.5 kDa to about 48 kDa.

In some embodiments, a recombinant protein disclosed herein comprises at least two repeating units, with each repeating unit comprising a degenerate molecular weight polypeptide standard having the same molecular weight of about 48 kDa to about 52 kDa. In aspects of these embodiments, a recombinant protein disclosed herein comprises at least two repeating units, with each repeating unit comprising a degenerate molecular weight polypeptide standard having the same molecular weight of about 48 kDa to about 50 kDa, or with each repeating unit comprising a degenerate molecular weight polypeptide standard having the same molecular weight of about 50 kDa to about 52 kDa. In other aspects of these embodiments, a recombinant protein disclosed herein comprises at least two repeating units, with each repeating unit comprising a degenerate molecular weight polypeptide standard having the same molecular weight of about 48 kDa to about 48.5 kDa, the same molecular weight of about 48.5 kDa to about 49 kDa, the same molecular weight of about 49 kDa to about 49.5 kDa, the same molecular weight of about 49.5 kDa to about 50 kDa, the same molecular weight of about 50 kDa to about 50.5 kDa, the same molecular weight of about 50.5 kDa to about 51 kDa, the same molecular weight of about 51 kDa to about 51.5 kDa, or the same molecular weight of about 51.5 kDa to about 52 kDa.

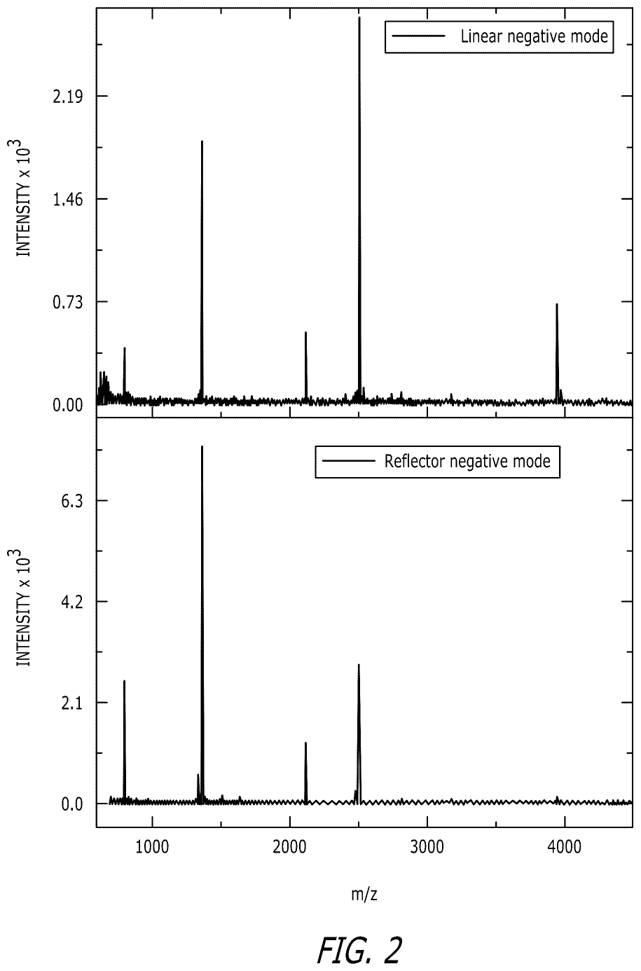

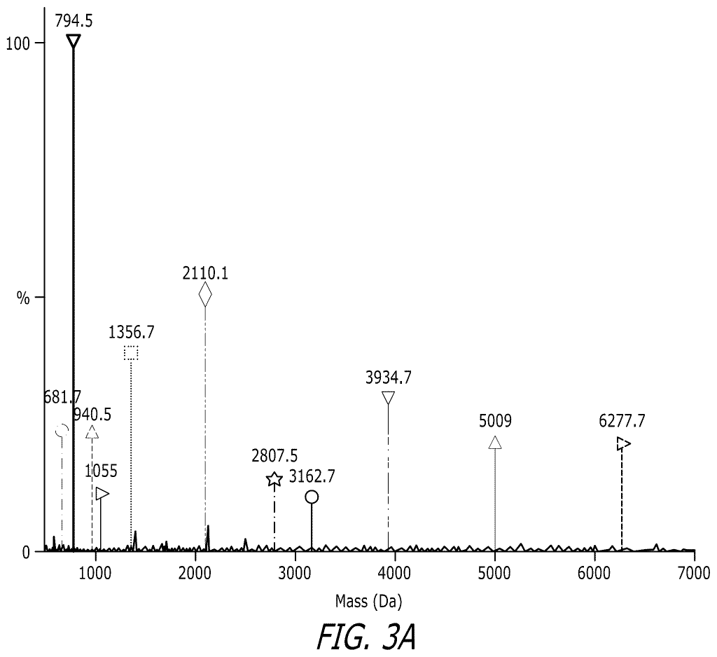

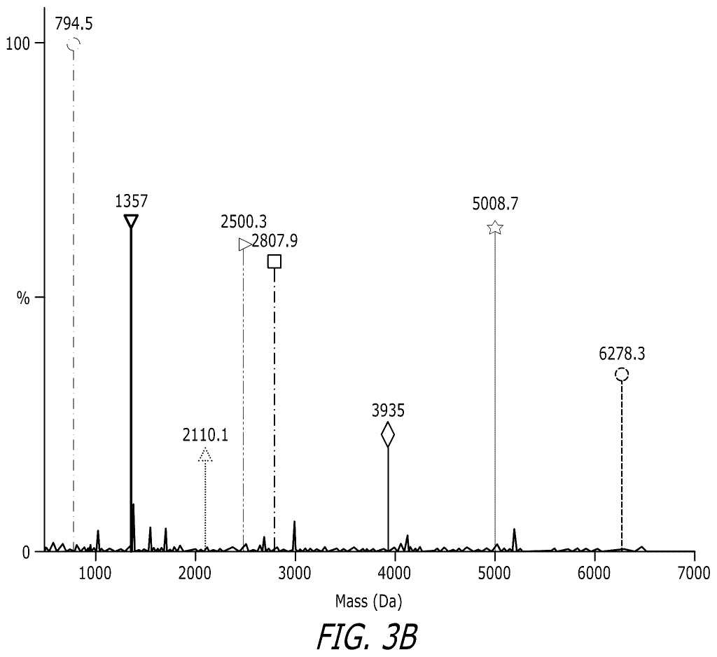

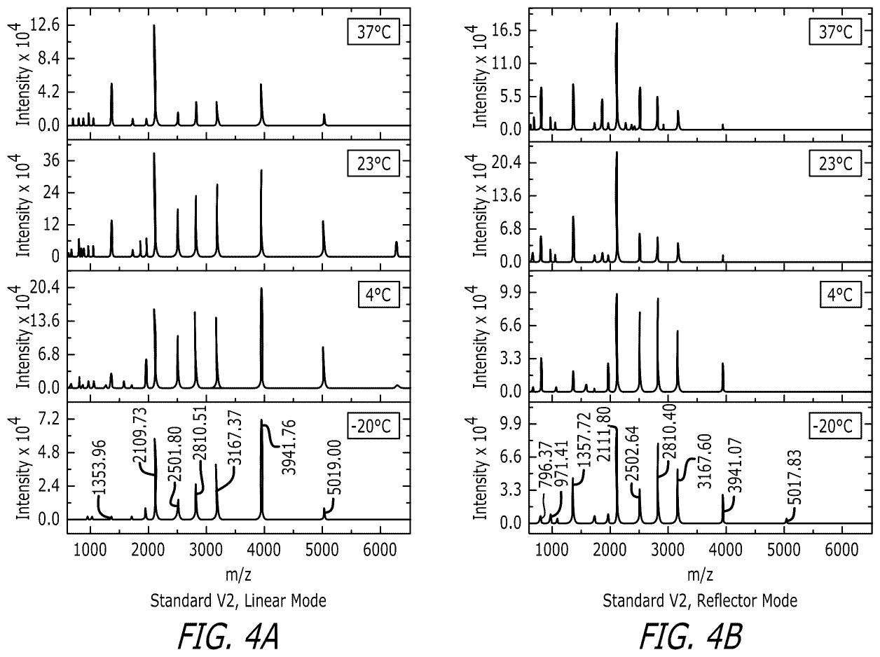

A recombinant protein and constituent molecular weight polypeptide standards disclosed herein are storage stable since both the recombinant protein and constituent molecular weight polypeptide standards exhibit high levels of stability when stored under various conditions. As disclosed in the Examples, stability of the recombinant protein and constituent molecular weight polypeptide standards was demonstrated by repeating MALDI-TOF analysis after a period of storage, after exposure to different numbers of freeze-thaw cycles, or in the presence of suitable solvents or matrix components used in MALDI-TOF. For example, incubation of the standard for eight months at −20° C., 4° C., room temperature (23° C.) and 37° C. did not lead to appreciable degradation of the peptides (FIG. 2). In addition, the samples were stable even on the MALDI plate in the presence of matrix for at least three days at room temperature indicating that such standars are storage stable in the presence of trifluoroacetic acid (FIG. 3). The molecular weight polypeptide standards were also resistant to exposure to multiple freeze thaw cycles (FIG. 4), showing minimal or no degradation after 5, 10 and 15 freeze-thaw cycles. These examples demonstrate that the molecular weight polypeptide standards disclosed herein are completely stable.

A recombinant protein and constituent molecular weight polypeptide standards disclosed herein will be stable (i.e. be substantially unchanged) after storage in solution (e.g. in the presence of 50% acetonitrile and 0.05% TFA) at any one of −20° C., 4° C., room temperature (23° C.) and 37° C. for at least 6 months, at least 9, months, at least 12 months, at least 15 months, at least 18 months, at least 21 months or at least 24 months. In addition, prepared samples mixed with a matrix composition on a MALDI plate are stable for at least 3 days, at least 4 days, at least 5 days, at least 6 days, at least 7 days or longer. Given that the standard solution contains 0.05% Trifluoroacetic acid, such a stability is unexpected. In one embodiment, the recombinant protein and polypeptide standards are storage stable in the presence of trifluoroacetic acid.

### Chemical Cleavage Site

Besides a molecular weight polypeptide standard, each repeating unit also includes a chemical cleavage recognition site. A chemical cleavage site disclosed herein is a chemical cleavage site cleaved by a site-specific proteolytic chemical agent. Suitable site-specific proteolytic chemical agents are known in the art. In some embodiments, the site-specific proteolytic chemical agent is selected from 2-nitro-5-thiocyanobenzoic acid (NTCB), Ni²⁺, Cu²⁺, Co²⁺ or Pd²⁺. In some embodiments, the site-specific proteolytic chemical agent is Ni²⁺.

### Repeating Units

A recombinant protein disclosed herein comprises a plurality of repeating units. The number of repeating units can vary according to the number of molecular weight polypeptide standards required. In one embodiment, n is an integer from 1 to 10, 1 to 15, 1 to 20, 1 to 25, 1 to 30, 1 to 35, 1 to 40, 1 to 45, 1 to 50, 2 to 10, 2 to 15, 1 to 20, 2 to 25, 2 to 30, 2 to 35, 2 to 40, 2 to 45, 2 to 50, 3 to 10, 3 to 15, 3 to 20, 3 to 25, 3 to 30, 3 to 35, 3 to 40, 3 to 45, 3 to 50, 4 to 10, 4 to 15, 4 to 20, 4 to 25, 4 to 30, 4 to 35, 4 to 40, 4 to 45, 4 to 50, 5 to 10, 5 to 15, 5 to 20, 5 to 25, 5 to 30, 5 to 35, 5 to 40, 5 to 45, 5 to 50, 6 to 10, 6 to 15, 5 to 20, 6 to 25, 6 to 30, 6 to 35, 6 to 40, 6 to 45, 6 to 50, 7 to 10, 7 to 15, 7 to 20, 7 to 25, 7 to 30, 7 to 35, 7 to 40, 7 to 45, 7 to 50, 8 to 10, 8 to 15, 8 to 20, 8 to 25, 8 to 30, 8 to 35, 8 to 40, 8 to 45, 8 to 50, 9 to 10, 9 to 15, 9 to 20, 9 to 25, 9 to 30, 9 to 35, 9 to 40, 9 to 45, 9 to 50, 10 to 15, 10 to 20, 10 to 25, 10 to 30, 10 to 35, 10 to 40, 10 to 45, 10 to 50, 15 to 20, 15 to 25, 15 to 30, 15 to 35, 15 to 40, 15 to 45, 15 to 50, 20 to 25, 20 to 30, 20 to 35, 20 to 40, 20 to 45, 20 to 50, 25 to 30, 25 to 35, 25 to 40, 25 to 45, or 25 to 50. In another embodiment, n is an integer from, e.g., 1 to 2, 1 to 3, 1 to 4, 1 to 5, 1 to 6, 1 to 7, 1 to 8, 1 to 9, 1 to 10, 2 to 3, 2 to 4, 2 to 5, 2 to 6, 2 to 7, 2 to 8, 2 to 9, 2 to 10, 3 to 4, 3 to 5, 3 to 6, 3 to 7, 3 to 8, 3 to 9, 3 to 10, 4 to 5, 4 to 6, 4 to 7, 4 to 8, 4 to 9, 4 to 10, 5 to 6, 5 to 7, 5 to 8, 5 to 9, 5 to 10, 6 to 7, 6 to 8, 6 to 9, 6 to 10, 7 to 8, 7 to 9, 7 to 10, 8 to 9, 8 to 10, or 8 to 10. In yet another embodiment, n is an integer from, e.g., 10 to 15, 10 to 20, 10 to 25, 10 to 30, 10 to 35, 10 to 40, 10 to 45, 10 to 50, 15 to 20, 15 to 25, 10 to 30, 15 to 35, 15 to 40, 15 to 45, 15 to 50, 20 to 25, 20 to 30, 20 to 35, 20 to 40, 20 to 45, 20 to 50, 25 to 30, 25 to 35, 25 to 40, 25 to 45, 25 to 50, 30 to 35, 30 to 40, 30 to 45, 30 to 50, 35 to 40, 35 to 45, 35 to 50, 40 to 45, 40 to 50, or 45 to 50.

### Tag

As previously indicated, a disclosed recombinant polypeptide herein may further comprise at least one tag. A tag may be present at the N-terminus or the C-terminus of the recombinant protein or, in some embodiments, at both the N-terminus and the C-terminus. When present, a tag does not form part of the molecular weight polypeptide standards. A tag may impart or increase any one or more desired functions on the recombinant polypeptide. Non limiting examples include, e.g., detection, purification, solubilization, protection from degradation, proper folding (chaperone activity), post-translational modification, N-terminal or C-terminal capping (capping units preferably are more hydrophilic than the rest of the protein and consequently shield the hydrophobic part to increase solubility (Kohl et al. (2003) Proc. Natl. Acad. Sci U.S.A. 100:1700-5)), receptor activity, signaling activity, secretion, and targeting. In particular examples, a disclosed recombinant polypeptide comprises a tag that facilitates purification (e.g., affinity purification) and/or solubilization of the polypeptide. In applications wherein a disclosed recombinant protein is to be secreted from the host cell, an appropriate signal peptide may be added to the polypeptide in order to direct the synthesized polypeptide to the secretion route of the host cell. Such signal peptides are known in the art, and heterologous signal peptides and signal peptides native to the host cell may generally be utilized. Non-limiting examples of the foregoing peptide tags are well known and commonly used in the art; e.g., thioredoxin (TrxA), His6, myc, T7, HSV, V5, HA, FLAG, strep-tags, GFP, chitin binding protein, GST, MBT, NusA, IF2, cellulose-binding module, barnase, IgG binding domain ZZ, GB1, and SUMO. Recombinant polypeptides for use in methods herein may be produced in any recombinant expression system; e.g., in cell culture, or in a cell-free system such as a cell lysate or coupled transcription/translation system, and then purified therefrom, for example and without limitation, by affinity purification with immobilized agents (e.g., small molecules, and antibodies) that bind a tag comprised within the polypeptide.

A tag can be adjacent to a molecular weight polypeptide standard or chemical cleavage site. In addition, as shown in Formulas Ill and IV, a tag is associated with a chemical cleavage site that enables removal of the tag. In some embodiments, the chemical cleavage site associated with a tag can be replaced by a cleavage site of a protease that leaves no extraneous amino acid at the P1′ position. For example, Table 2 discloses cleavage sites recognized and cleaved by a protease that leaves no extraneous amino acid at the P1′ position.

TABLE 2
Proteases Leaving No Amino Acid at
the P1′ Position Following Cleavage

| | Recognition | SEQ |
|---|---|---|
| Protease | Site* | ID NO: |
| Canonical Protease | (Y/F/W/D/I/L/T/V)(E/ | 1 |
| Recognition Site | D/V)X(D/R/K)↓ | |
| Enterokinase | DDDDK↓ | 2 |
| Caspase Recognition Site | (Y/F/W/D/I/L/T/V)(E/V)XD↓ | 3 |
| Caspase-2, -3, -7, CED-3 | DEXD↓ | 4 |
| Caspase-1, -4, -5 | (Y/F/W)VXD↓ | 6 |
| Caspase-6, -8, -9, | (I/L/T/V)EXD↓ | 10 |
| and Granzyme B | | |
| Factor Xa | I(E/D)GR↓ | 14 |

*Amino acids immediately preceding cleavage site (e.g., P4-P3-P2-P1↓, P5-P4-P3-P2-P1↓, etc.)

In some embodiments, the chemical cleavage site closest to the tag is replaced by a protease cleavage site. For example, the protease cleavage site can be a site recognized and cleaved by a caspase, e.g., caspase-3 or caspase-7. In some embodiments, the protein comprises a sequence selected from SEQ ID NO: 59 or SEQ ID NO: 86.

### Method of Production

The present specification also provides a method of producing a plurality of molecular weight polypeptide standards. The disclosed method can comprise: a) expressing a recombinant protein arranged according to formulas I, II, III, or IV disclosed herein; b) isolating the expressed recombinant protein; and c) cleaving the chemical cleavage site or sites of the isolated recombinant protein using a chemical agent that recognizes the chemical cleavage site to produce a plurality of molecular weight polypeptide standards, wherein each molecular weight polypeptide standard has its amino acid sequence without any extraneous amino acids.

Expression of a recombinant protein disclosed herein can be achieved using a standard expression system known in the art and useful for expressing a recombinant protein disclosed herein. Cell-based systems include, without limitation, viral expression systems, prokaryotic expression systems, yeast expression systems, baculoviral expression systems, insect expression systems and mammalian expression systems. Expression of a recombinant protein disclosed herein using a cell-base system generally employs a host cell that is capable of having an expression construct disclosed herein introduced into, and transient or stably maintained within, the host cell and a culture system for that host cell.

Cell-free systems include, without limitation, prokaryotic extracts and eukaryotic extracts such as, e.g., wheat germ extracts, rabbit reticulocyte extracts and E. coli extracts. Expression of a recombinant protein disclosed herein using a cell-base system generally employs an in vitro system used to express an expression construct disclosed herein in order to produce an encoded recombinant protein disclosed herein.

Isolation of the expressed recombinant protein disclosed herein can be achieved using well-established purification techniques known in the art such as, e.g., affinity chromatography, high-performance liquid chromatography (HPLC), capillary electrophoresis, capillary electrochromatography, and size-exclusion chromatography.

In some embodiments, the cleaving of step c) results in complete digestion of a recombinant protein disclosed herein to produce the molecular weight polypeptide standards. In some embodiments, the cleaving of step c) results in partial digestion of a recombinant protein disclosed herein to produce the molecular weight polypeptide standards. This results in two or more repeating units remaining connected and can allow for the provision of longer, higher molecular weight standard proteins without the need to include an individual repeating unit providing a higher molecular weight within the recombinant protein as a whole. Use of partial digestion provides improved flexibility and can reduce the overall size of the recombinant protein to be expressed. For example, larger molecular weight polypeptide standards can be produced without requiring the recombinant protein as a whole to exceed the capacity of a given vector-host cell system. For example, a recombinant protein with the molecular weight of approximately 194 kDa can be designed to produce a set of molecular weight polypeptide standards that covers a range from 3.5 kDa to 34 kDa with 12 calibration points. Production of a protein of this size cannot generally be accomplished in E. coli due to its size and even if the recombinant protein can be produced the yield is extremely low. However, partial digestion can overcome this issue, such that a 34 kDa protein can produce 12 or even more fragments. It is of note that proteins smaller than 50 kDa are produced in E. coli with very high yields.

Use of a chemical agent is advantageous when employing a step involving partial digestion of a recombinant protein disclosed herein because such digestion cannot readily be achieved using proteolytic enzymatic cleavage of the recombinant protein. For example, proteases are extremely efficient enzymes that cleave their recognition sites quickly making partial digestion extremely difficult to reproducibly achieve. In addition, the high turnover number of proteases exacerbate the difficulties in controlling partial digestion in a manner that results in consistent and reproducible outcomes. In addition, the peptide sequences cannot contain internal Arg or Lys residues, basic amino acids that adversely affect the performance of molecular weight polypeptide standards used in MALDI. However, the use of chemical cleavage with ions such as Ni or Pd allows precise control of partial digestion reducing batch to batch variability of the product due to low turnover of the reactions by these agents and the fact that complete digestion takes hours to achieve. In addition, the cleavage recognition site of a chemical can be modified in order to enhance or retard cleavage at that site. For example, SXH is the Ni recognition sequence. By dictating the residue designated as X as well as the amino acid flanking the carboxyl end of histidine, Ni will more or less effectively hydrolyze this site. Thus, the recognition sequence SWHR is efficiently cleaved by NI while the recognition site SIHV is poorly hydrolyzed by Ni. This allows for a recombinant protein disclosed herein have an overall lower molecular weight while still producing the same range of molecular weight polypeptide standards. Thus, a 60 kDa recombinant protein having six repeating units where each molecular weight polypeptide standard is 10 kDa can produce standards of 60 kDa, 50 kDa, 40 kDa, 30 kDa, 20, Kda and 10 kDa through partial digestion. Reliance on a complete digestion scheme would require a recombinant protein of 210 kDa in order to achieve the same range of molecular weight polypeptide standards. As construction, expression and processing of larger proteins is more difficult and variable simply due to their size relative to smaller proteins, partial digestion is an elegant way to achieve the same desired result. Accordingly, the use of chemical cleavage can provide greater flexibility, efficiency and consistency.

When a protease cleavage site is present, in some embodiments the method comprises after step b) and before step c) the additional steps: i) cleaving the protease cleavage site with a protease to cleave the tag from the recombinant protein; and ii) purifying the products of step i) to remove the cleaved tag and the protease.

In some embodiments, a host cell is transfected or transformed with an expression or cloning vector comprising a polynucleotide encoding the recombinant protein (e.g., a polypeptide that is soluble in the cytosol of the cell), and the host cell is cultured in a conventional nutrient medium. Culture conditions, such as solute composition, temperature, and pH, can be selected from any of the many conditions known to support growth of particular host cells. In general, principles, protocols, and practical techniques for maximizing cell culture productivity are well-known and widely available to those in the art.

Aspects of the present specification can also be described by the following embodiments:

1. A recombinant protein comprising one or more repeating units, each of the one or more repeating units comprising a chemical cleavage site and a molecular weight polypeptide standard, wherein the chemical cleavage site is an amino acid sequence, the recombinant protein arranged according to formula I,

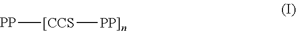

wherein brackets indicate a repeating unit of the one or more repeating units; PP is a molecular weight polypeptide standard; CCS is a chemical cleavage site; and n is an integer from 1 to 50.

2. The recombinant protein of embodiment 1, wherein the chemical cleavage site is a chemical cleavage site cleaved by a site-specific proteolytic chemical agent.

3. The recombinant protein of embodiment 2, wherein the site-specific proteolytic chemical agent is selected from 2-nitro-5-thiocyanobenzoic acid (NTCB), Ni²⁺, Cu²⁺, Co²⁺ or Pd²⁺.

4. The recombinant protein of embodiment 3, wherein the site-specific proteolytic chemical agent is Ni²⁺.

5. The recombinant protein of any one of embodiments 1-4, wherein n is an integer from 5 to 25.

6. The recombinant protein of any one of embodiments 1-5, wherein n is an integer from 5 to 15.

7. The recombinant protein of any one of embodiments 1-6, wherein the repeating units comprise different molecular weight polypeptide standards, each having a determined molecular weight.

8. The recombinant protein of embodiment 6, wherein two or more of the different molecular weight polypeptide standards each have a different determined molecular weight.

9. The recombinant protein of embodiment 6, wherein two or more of the different molecular weight polypeptide standards each have the same determined molecular weight but a different amino acid sequence.

10. The recombinant protein of any one of embodiments 1-9, wherein the recombinant protein comprises formula II or formula III:

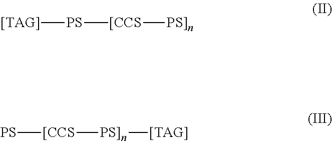

wherein brackets indicate a repeating unit of the one or more repeating units; PP is the product peptide; CCS is the chemical cleavage site; TAG is the affinity purification tag or the solubilization tag; and n is an integer from 1 to 50.

11. The recombinant protein of any one of embodiments 1-10, wherein the recombinant protein comprises formula IV or formula V:

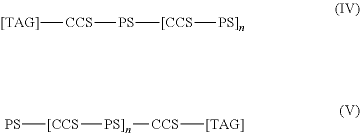

wherein brackets indicate a repeating unit of the one or more repeating units; PP is the product peptide; CCS is the chemical cleavage site; TAG is the affinity purification tag or the solubilization tag; and n is an integer from 1 to 50.

12. The recombinant protein of embodiment 11, wherein the chemical cleavage site closest to the tag is replaced by a protease cleavage site.

13. The recombinant protein of any one of embodiments 1-12, wherein the protein is storage stable in the presence of trifluoroacetic acid.

14. The recombinant protein of any one of embodiments 1-13, wherein the protein comprises a sequence selected from SEQ ID NO: 59 or SEQ ID NO: 86.

15. A nucleic acid encoding the recombinant polypeptide of any one of embodiments 1-14.

16. An expression construct comprising an expression vector including the nucleic acid of embodiment 15.

17. An expression system comprising an expression construct of embodiment 16.

18. The expression system of embodiment 17, wherein the expression system is a cell-based system or a cell-free system.

19. A host cell comprising the nucleic acid of embodiment 15 or the expression of embodiment 16.

20. The host cell of embodiment 19, wherein the host cell is selected from a bacterial cell, a fungal cell, an insect cell, a mammalian cell or a plant cell.

21. A method of producing a plurality of molecular weight polypeptide standards, the method comprising: a) expressing a recombinant protein from a nucleic acid sequence encoding the recombinant protein, the recombinant protein comprising one or more repeating units, each of the one or more repeating units comprising a chemical cleavage site and a molecular weight polypeptide standard, wherein the chemical cleavage site is an amino acid sequence, the recombinant protein arranged according to formula I

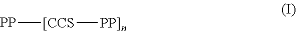

wherein brackets indicate a repeating unit of the one or more repeating units; PP is a molecular weight polypeptide standard; CCS is a chemical cleavage site; and n is an integer from 1 to 50; b) isolating the expressed recombinant protein; c) cleaving the chemical cleavage site or sites of the isolated recombinant protein using a chemical agent that recognizes the chemical cleavage site to produce a plurality of molecular weight polypeptide standards, wherein each molecular weight polypeptide standard has its amino acid sequence without any extraneous amino acids.

22. The method of embodiment 21, wherein the chemical cleavage site is a chemical cleavage site cleaved by a site-specific proteolytic chemical agent.

23. The method of embodiment 22, wherein the site-specific proteolytic chemical agent is selected from 2-nitro-5-thiocyanobenzoic acid (NTCB), Ni²⁺, Cu²⁺, Co²⁺ or Pd²⁺.

24. The method of embodiment 23, wherein the site-specific proteolytic chemical agent is Ni²⁺.

25. The method of any one of embodiments 21-24, wherein the cleaving of step c) results in partial digestion of the recombinant protein.

26. The method of any one of embodiments 21-25, wherein the cleaving of step c) results in complete digestion of the polypeptide.

27. The method of any one of embodiments 21-26, wherein n is an integer from 5 to 25.

28. The method of embodiment 27, wherein n is an integer from 5 to 15.

29. The method of any one of embodiments 21-28, wherein the repeating units comprise different molecular weight polypeptide standards, each having a determined molecular weight.

30. The method of embodiment 29, wherein two or more of the different molecular weight polypeptide standards each have a different determined molecular weight.

31. The method of embodiment 29, wherein two or more of the different molecular weight polypeptide standards each have the same determined molecular weight but a different amino acid sequence.

32. The method of any one of embodiments 21-32, wherein the recombinant protein comprises formula II or formula III:

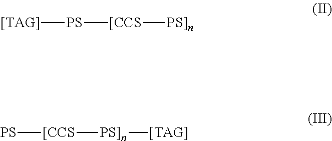

wherein brackets indicate a repeating unit of the one or more repeating units; PP is the product peptide; CCS is the chemical cleavage site; TAG is the affinity purification tag or the solubilization tag; and n is an integer from 1 to 50.

33. The method of any one of embodiments 21-32, wherein the recombinant protein comprises formula IV or formula V:

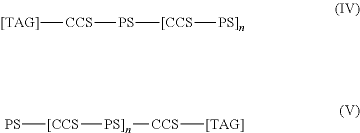

wherein brackets indicate a repeating unit of the one or more repeating units; PP is the product peptide; CCS is the chemical cleavage site; TAG is the affinity purification tag or the solubilization tag; and n is an integer from 1 to 50.

34. The recombinant protein of embodiment 33, wherein the chemical cleavage site closest to the tag is replaced by a protease cleavage site.

35. The method of embodiment 34, comprising after step b) and before step c) the additional steps: i) cleaving the protease cleavage site with a protease to cleave the tag from the recombinant protein; and ii) purifying the products of step i) to remove the cleaved tag and the protease.

36. A set of molecular weight polypeptide standards obtainable by the method of any one of embodiments 21-35.

37. Use of the set of molecular weight standards of embodiment 36 for calibrating a MALDI-TOF mass spectrometer.

### EXAMPLES

The following non-limiting examples are provided for illustrative purposes only in order to facilitate a more complete understanding of representative embodiments now contemplated. These examples should not be construed to limit any of the embodiments described in the present specification, including those pertaining to the compounds, pharmaceutical compositions, or methods and uses disclosed herein.

### Example 1: Exemplary Degenerative Molecular Weight Polypeptide Standards

It is desirable to prevent any recombination during the expression of protein or gene synthesis. In order to achieve this, the following strategy was applied with reference to an exemplary polypeptide with mono isotopic mass of 1721.906 Da. The following sequence SRHSPSLSASGALPVLSG (SEQ ID NO: 71) would produce the intended mass. There are other degenerate sequences like SRHSVAPLGSASLLSPSG (SEQ ID NO: 72) and SRHSPSLSASGALPVLSG (SEQ ID NO: 73) that would also produce the exact same mass, but differ in the order of the amino acids. The SRHS sequence (SEQ ID NO: 100-Ni recognition sequence for hydrolysis) remained fixed but degenerate sequences were used for the remainder of the sequence. Thus, the construct had three repeats of sequences that would yield 1721.906 Da. For smaller masses, in addition to using degenerate sequences, the sequences can be paced apart from each other, i.e., distributed along the length of the protein to minimize the repetitiveness. These precautions prevent recombination inside the host, in this case E. coli. As a result of this use of degenerate sequences, the expression construct was absolutely stable during the production.

Their cDNAs were cloned in pET28a vector, then the protein was expressed in E. coli. Following purification of the protein, it was completely digested by nickel ions for standards that are used for masses below 6500 Da, and partially digested for masses above 6500 Da. Then the proteins were subjected to HPLC (C18 reverse phase.

The number of polypeptides incorporated in the construct that produce the exact same mass in the recombinant protein was dependent upon the intensity of the peak in MALDI-TOF instrument. Smaller polypeptides tend to fly better than larger polypeptides. Therefore, more of the larger polypeptides were included in the final recombinant protein sequence. For example, for the intended mass of 3161.7502 Da, with the sequence of SRHSPALLPASVGAVAVFLFRSPFGLAAPGLG (SEQ ID NO: 82), three more degenerate sequences of SRHSLSLAVVFSFPRAGAVFGPAALLAPLPGG (SEQ ID NO: 83), SRHSLRVFPSAFFPLGVGLPASGLAALPAVAG (SEQ ID NO: 84), and SRHSPSGFVAVAPRLFAPVLFPSGLALAGALG (SEQ ID NO: 85) were used, while for the mass of 968.5403 a total of three degenerate peptide sequences was used.

### Example 2: Exemplary Recombinant Protein

Exemplary proteins according to the present specification and constituent molecular weight polypeptide standards are provided in Table 3. In the first example, recombinant protein of SEQ ID NO: 59 (shown below) has a molecular weight of 48.27 kDa, and a pI of 14.01. This recombinant protein is cleaved using Ni²⁺ ions to produce molecular weight polypeptide standards SEQ ID NOs: 62-85 (Table 3). SEQ ID NO: 59 includes a N-terminal His6 purification tag followed by a recognition sequence for caspase-3 (SEQ ID NO: 60) or caspase-7 (SEQ ID NO: 61) to allow cleavage of the tag and its subsequent removal during HPLC. i.e., the tag is not one of the molecular weight polypeptide standards.

MGSSHHHHHHSSGLVPRGSHMASMTGGQQMGRG⋄SRHLLG⋄SRHSPSLSA
SGALPVLSG⋄SRHSLSLLG⋄SRHSVSPPLSFSG⋄SRHLLG⋄SRHSLLS
LGSRHSPVSSFPLSG⋄SRHLLG⋄SRHSVAPLGSASLLSPSG⋄SRHSLL
LSG⋄SRHSPSLPSVFSG⋄SRHSISGSSGVPRAPFAPGSVG⋄SRHSIVF
PSGSGPSVGAPRASG⋄SRHSPSLLSFAPSGAVLSPFSVLSG⋄SRHSGSL
PSPLASLSFVALPSSFVG⋄SRHSPSLLSFAPSGAVLSPFSVLSG⋄SRHS
ALRVPVPLFGPALASLFAGSGFVGSRHSLPSGFAFVAAVLLRVAFSPGL
GPG⋄SRHSGAAAFFRSLPVGLVVAFSLPGLPG⋄SRHSPALLPASVGAVA
VFLFRSPFGLAAPGLG⋄SRHSLSLAVVFSFPRAGAVFGPAALLAPLPGG
⋄SRHSLRVFPSAFFPLGVGLPASGLAALPAVAG⋄SRHSPSGFVAVAPRL
FAPVLFPSGLALAGALG

In the second example, recombinant protein of SEQ ID NO: 86 (shown below) has a molecular weight of 34.2967 kDa, and a pI of 13.05. This recombinant protein is cleaved using Ni²⁺ ions to produce molecular weight polypeptide standards SEQ ID NOs: 89-96. SEQ ID NO: 86 also includes a N-terminal His6 purification tag followed by a recognition sequence for caspase-3 (SEQ ID NO: 87) or caspase-7 (SEQ ID NO: 88) to allow cleavage of the tag and its subsequent removal during HPLC. i.e., the tag is not one of the molecular weight polypeptide standards.

MGSSHHHHHHSSGLVPRGSHMASMTGGQQMGRGSDEVDG⋄SRHLLLG⋄SR
HSPSPSLFSVG⋄SRHSISGSSGVPRAPFAPGSVGLSIVFPSGSGPSVGAP
RASG⋄SRHSPSLLSFAPSGAVLSPFSVLSGLRHSGSLPSPLASLSFVALP
SSFVG⋄SRHSPSLLSFAPSGAVLSPFSVLSGLHG⋄SRHSPSLLSFAPSGA
VLSPFSVLSGLHSGSLPSPLASLSFVALPSSFVG⋄SRHSPALLPASVGAV
AVFLFRSPFGLAAPGLGLSLSLAVVFSFPRAGAVFGPAALLAPLPGG⋄SR
HSLRVFPSAFFPLGVGLPASGLAALPAVAGLRHSPSGFVAVAPRLFAPVL
FPSGLALAGAL

### Davoodi, Molecular Weight Standards

TABLE 3
Recombinant Polypeptides

| Monoisotopic | Average Mass | | |
|---|---|---|---|
| Mass (Da) | (Da) | Sequence | SEQ ID NO: |
| 3′245.4275 | 3′247.491 | SHHHHHHSSGLVPRGSHMASMTGGQQMGRG | 60 |
| 3332.4748 | 3′334.5684 | SSHHHHHHSSGLVPRGSHMASMTGGQQMGR | 61 |
| | | G | |
| 681.3922 | 681.7853 | SRHLLG | 62 |
| | | SRHLLG | 63 |
| | | SRHLLG | 64 |
| 968.5403 | 969.098 | SRHSLSLLG | 65 |
| | | SRHSLLSLG | 66 |
| | | SRHSLLLSG | 67 |
| 1356.6786 | 1357.4733 | SRHSVSPPLSFSG | 68 |
| | | SRHSPVSSFPLSG | 69 |
| | | SRHSPSLPSVFSG | 70 |
| 1721.906 | 1722.8997 | SRHSPSLSASGALPVLSG | 71 |
| | | SRHSVAPLGSASLLSPSG | 72 |
| | | SRHSPSLSASGALPVLSG | 73 |
| 2109.0715 | 2110.2935 | SRHSISGSSGVPRAPFAPGSVG | 74 |
| | | SRHSIVFPSGSGPSVGAPRASG | 75 |
| 2499.3122 | 2500.8075 | SRHSPSLLSFAPSGAVLSPFSVLSG | 76 |
| | | SRHSGSLPSPLASLSFVALPSSFVG | 77 |
| | | SRHSPSLLSFAPSGAVLSPFSVLSG | 78 |
| 2809.5392 | 2811.248 | SRHSALRVPVPLFGPALASLFAGSGFVG | 79 |
| | | SRHSLPSGFAFVAAVLLRVAFSPGLGPG | 80 |
| | | SRHSGAAAFFRSLPVGLVVAFSLPGLPG | 81 |
| 3161.7502 | 3163.6773 | SRHSPALLPASVGAVAVFLFRSPFGLAAPGLG | 82 |
| | | SRHSLSLAVVFSFPRAGAVFGPAALLAPLPGG | 83 |
| | | SRHSLRVFPSAFFPLGVGLPASGLAALPAVAG | 84 |
| | | SRHSPSGFVAVAPRLFAPVLFPSGLALAGALG | 85 |
| 3′847.6459 | 3′850.0404 | SHHHHHHSSGLVPRGSHMASMTGGQQMGRG | 87 |
| | | SDEVDG | |
| 3934.6779 | 3937.1178 | SSHHHHHHSSGLVPRGSHMASMTGGQQMGR | 88 |
| | | GSDEVDG | |
| 794.4763 | 794.9432 | SRHLLLG | 89 |
| 1356.6786 | 1357.4733 | SRHSPSPSLFSVG | 90 |
| 3933.0245 | 3935.3268 | SRHSISGSSGVPRAPFAPGSVGLSIVFPSGSGP | 91 |
| | | SVGAPRASG | |
| 5006.6658 | 5009.6801 | SRHSPSLLSFAPSGAVLSPFSVLSGLRHSGSLP | 92 |
| | | SPLASLSFVALPSSFVG | |
| 2806.4766 | 2808.1563 | SRHSPSLLSFAPSGAVLSPFSVLSGLHG | 93 |
| 4850.5647 | 4853.4942 | SRHSPSLLSFAPSGAVLSPFSVLSGLHSGSLPS | 94 |
| | | PLASLSFVALPSSFVG | |
| 6038.3819 | 6042.0943 | SRHSPALLPASVGAVAVFLFRSPFGLAAPGLGL | 95 |
| | | SLSLAVVFSFPRAGAVFGPAALLAPLPGG | |
| 6274.5204 | 6278.3683 | SRHSLRVFPSAFFPLGVGLPASGLAALPAVAGL | 96 |
| | | RHSPSGFVAVAPRLFAPVLFPSGLALAGAL | |

### Davoodi, Molecular Weight Standards

It will be appreciated that there is scope for generalization of the formula for the recombinant protein, which can be provided as: TAG-(G or A or S)-S-(R or K)—H-Seq1-(G or A or S)-S-(R or K)—H-Seq2 (G or A or S)-S-(R or K)—H-Seq3 (G or A or S)-S-(R or K)—H-Seq4 (G or A or S)-S-(R or K)—H-Seq5 (G or A or S), in which SXH is the Ni recognition sequence. A tag can be added at the C-terminus, providing the following general formula: Met-(G or A or S)-S-(R or K)—H-Seq1-(G or A or S)-S-(R or K)—H-Seq2 (G or A or S)-S-(R or K)—H-Seq3 (G or A or S)-S-(R or K)-H-Seq4 (G or A or S)-S-(R or K)—H-Seq5 (G or A or S)S-(R or K)—H-Tag.

When expressed in E. coli the initiator methionine is removed inside E coli.

The above sequence is suitable for both negative and positive mode MALDI-TOF. Under some circumstances, particularly for the negative mode, the sequence can be modified as follows: TAG-(G or A or S)-S-(D or E)-H-Seq1-(G or A or S)-S-(D or E)-H-Seq2 (G or A or S)-S-(D or E)-H-Seq3 (G or A or S)-S-(D or E)-H-Seq4 (G or A or S)-S-(D or E)-H-Seq5 (G or A or S) or Met-(G or A or S)-S-(D or E)-H-Seq1-(G or A or S)-S-(D or E)-H-Seq2 (G or A or S)-S-(D or E)-H-Seq3 (G or A or S)-S-(D or E)-H-Seq4 (G or A or S)-S-(D or E)-H-Seq5 (G or A or S)S-(D or E)-H-Tag.

### Example 3: Production of Molecular Weight Standards

The following example illustrated how to produce molecular weight polypeptide standards disclosed herein from a recombinant protein disclosed herein.

An expression construct encoding recombinant protein SEQ ID NO: 59 or SEQ ID NO: 86, was transformed into and expressed in E coli to obtain the recombinant protein. The cells were then lysed using guanidine hydrochloride, and the expressed recombinant protein were purified by Ni-NTA Sepharose. The solution containing the purified recombinant protein was then purged with air for few minutes in order to saturate the solution with oxygen in order to oxidize methionine into its oxidized form. The purified recombinant protein was then incubated at 40° C. to 50° C. overnight in a solution comprising 50 mM Hepes buffer (pH 8.2), 120 mM NaCl, and 1-2 mM of NiCl₂ (NiSO₄ can also be used) in order to cleave the recombinant protein and convert it into the desired molecular weight polypeptide standards. The incubation can also be carried out at room temperature and the duration can be shortened to only a few hours. Complete hydrolysis of these proteins by Ni²⁺ ions resulted in production of the molecular weight polypeptide standards listed above in Table 3.

In an alternative embodiment, the purified recombinant protein can be chemically cleaved using Pd²⁺ ions. In this case, the purified recombinant protein was then incubated at 60° C. overnight equimolar amount of cis-[Pd-(en)(H₂O)₄]²⁺ in order to cleave the recombinant protein and convert it into the desired molecular weight polypeptide standards. Complete hydrolysis of these proteins by Pd²⁺ ions resulted in production of the molecular weight polypeptide standards listed above in Table 3.

The chemically cleavage solution was then incubated with caspase-3 (SEQ ID NO: 59) or caspase-7 (SEQ ID NO: 86) to cleave the His6 purification tag and enable its subsequent removal. In one series of experiments, 5 mg of purified, chemically cleavage, polypeptide was added to a solution of 50 mM HEPES, pH 7.4, 100 mM NaCl, 10% glycerol, 0.1 mM EDTA, and 5 mM BME to which 50 μg of recombinant histidine tagged Caspase-3 or Caspase-7 protease was added and incubated overnight at 30° C., cleaving the polypeptide comprising the caspase-3 (SEQ ID NO: 59) or caspase-7 (SEQ ID NO: 86) recognition site.

In an alternative embodiment, proteolytic cleavage of the tag from the recombinant protein can be performed first with subsequent chemical cleavage by Ni²⁺ or Pd²⁺ ions to produce the molecular weight polypeptide standards.

In this case, the purified recombinant protein is first digested with Caspase-3 or Caspase-7 to cleave the polypeptide at the Caspase-3/Caspase-7 recognition site, and the tag and caspase are removed with a Ni-NTA column, due to the presence of six histidine residues present in both caspase and the His6 purification tag. Next, the resulting proteolytically cleaved polypeptide is treated with Ni²⁺ or Pd²⁺ ions as discussed above, to produce the molecular weight polypeptide standards listed above in Table 3.

After chemical and proteolytic digestion, the molecular weight polypeptide standards were precipitated by centrifugation, the pellet dissolved in 5% Acetonitrile and 0.1% Trifluoroacetic acid (TCA), and a sample analyzed by reverse phase HPLC using C18 columns. The recombinant protein and its molecular weight polypeptide standards were eluted at 20.2 and 20.4 min.

Following chemical and proteolytic digestion, MALDI-TOF analysis of the fractions was performed. Most fractions contained peptides with different masses. Based on the intended coverage mass range and number of peaks per standard, the fractions were mixed. Thus, all versions of the standards (i. e. version V1 through V6) are alterations of mixtures of these fractions. That is, the different versions contained the same polypeptides in differing proportions.

### Example 4: Analysis of Molecular Weight Polypeptide Standards

Molecular weight polypeptide standards were produced using the methods described in Examples 1 and 2 in order to calibrate a MALDI-TOF mass spectrometer.

The resultant molecular weight polypeptide standards described in Examples 1 and 2 were analyzed by MALDI-TOF in linear and reflector modes as well as positive and negative modes. The following are the images of two standards version 5 and 6 in both linear and reflector modes: Please note that a), b) the differences in various versions of standards (versions 2, 5, and 6 that are discussed here) are in the concentrations of the masses included in each standard, c) the eight months stability results for the Version 2 calibration standard are applicable to other standards, because the peptidic building blocks are the same except for the quantity of masses.

Stability is indicated by the continued detection of peaks indicative of the correct molecular weights and/or the absence of additional or altered peaks indicative of the presence of degradation products and/or alteration in molecular weight of the standard polypeptides.

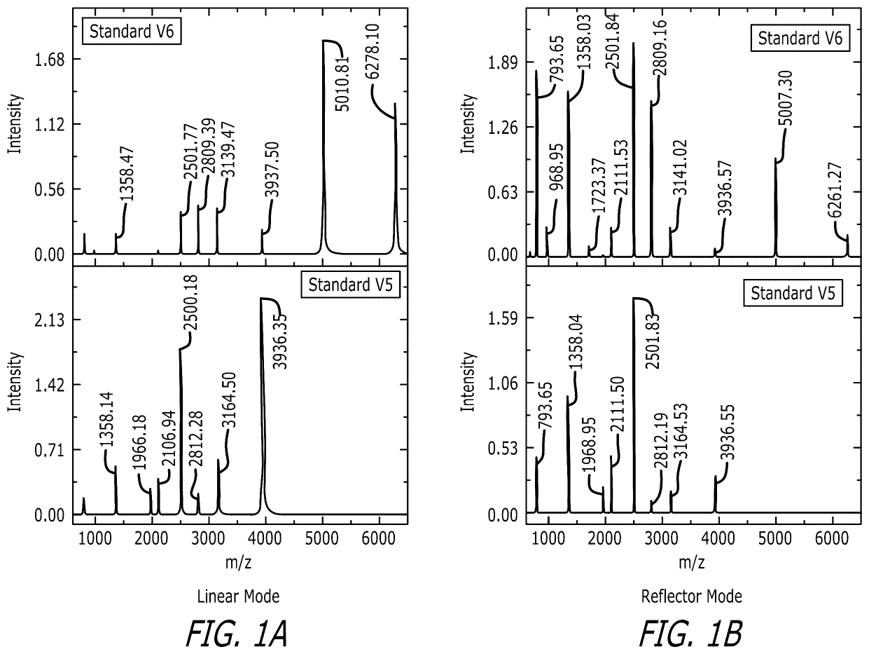

FIGS. 1A & 1B are exemplary spectra showing the detection peaks for each of the molecular weight polypeptide standards from version 5 (V5) or 6 (V6) of a recombinant protein disclosed herein. FIG. 1A shows spectra analyzed using a linear positive mode. This spectral analysis reveals seven distinct peaks were detected in molecular weight polypeptide standards V5 as well as molecular weight polypeptide standards V6. FIG. 1B shows spectra analyzed using a reflector positive mode. This spectral analysis exhibited eight distinct peaks for molecular weight polypeptide standards V5 and eleven distinct peaks for molecular weight polypeptide standards V6.

MALDI-TOF analysis for molecular weight polypeptide standards V5 was repeated in linear and reflector modes but operated in the negative mode. FIG. 2 is exemplary spectra showing the detection peaks for each of the molecular weight polypeptide standards V5, with five peaks identified in the linear negative mode and four peaks detected in the reflector negative mode. These data combined with the spectra from the same sample in the positive modes (linear and reflector) clearly show that the standard can be used to calibrate MALDI-TOF mass spectrometer both in negative and positive modes.

In order to show that correct molecular weight polypeptide standards are created by Ni hydrolysis and that these polypeptides do not undergo unwanted modifications during the various stages of processing and production, two different versions (V3 and V6) of the molecular weight polypeptide standards were analyzed by Q-Exactive mass spectrometer, a device that is extremely precise in determining the masses of polypeptides. The results for molecular weight polypeptide standards V3 are shown in FIG. 3A and those for molecular weight polypeptide standards V5 are shown in FIG. 3B. It can be seen that the molecular weight polypeptide standards V3 and V6 produce the same peaks, but at different intensities in accordance with the different relative proportions of polypeptides in each version. For example, FIG. 3A exhibits eleven peaks for molecular weight polypeptide standards V3 while FIG. 3B exhibits eight peaks for molecular weight polypeptide standards V6. The eight peaks detected for molecular weight polypeptide standards V6 are also present in molecular weight polypeptide standards V3, with the latter detecting three additional peaks.

### Example 5: Stability of Molecular Weight Polypeptide Standards

Stability of the molecular weight polypeptide standards when stored under various conditions was assessed by repeating the initial MALDI-TOF analysis of Example 3 after a period of storage or after exposure to different numbers of freeze-thaw cycles. Stability is indicated by the continued detection of peaks indicative of the correct molecular weights of the peptides and/or the absence of additional or altered peaks indicative of the presence of degradation products and/or alteration in molecular weight of the standard polypeptides. The analysis revealed that the molecular weight polypeptide standards disclosed herein exhibited high levels of stability and resistance to degradation under all conditions tested.

Long-term stability of the molecular weight polypeptide standards was assessed by repeating the initial MALDI-TOF analysis of Example 3 after a period of storage at various temperatures. In one series of experiments, a solution of 50% acetonitrile, 0.05% TFA, and exemplary molecular weight polypeptide standards V2 was stored for eight months at temperatures of at −20° C., 4° C., room temperature (23° C.), and 37° C. Exemplary MALDI-TOF spectra analyzed using a linear positive mode (FIG. 4A) and a reflector positive mode (FIG. 4B) reveal that incubation of molecular weight polypeptide standards V2 did not lead to appreciable degradation of the peptides at all temperatures tested after eight months of storage indicating that long-term stability of molecular weight polypeptide standards V2 was maintained over a wide range of temperatures.

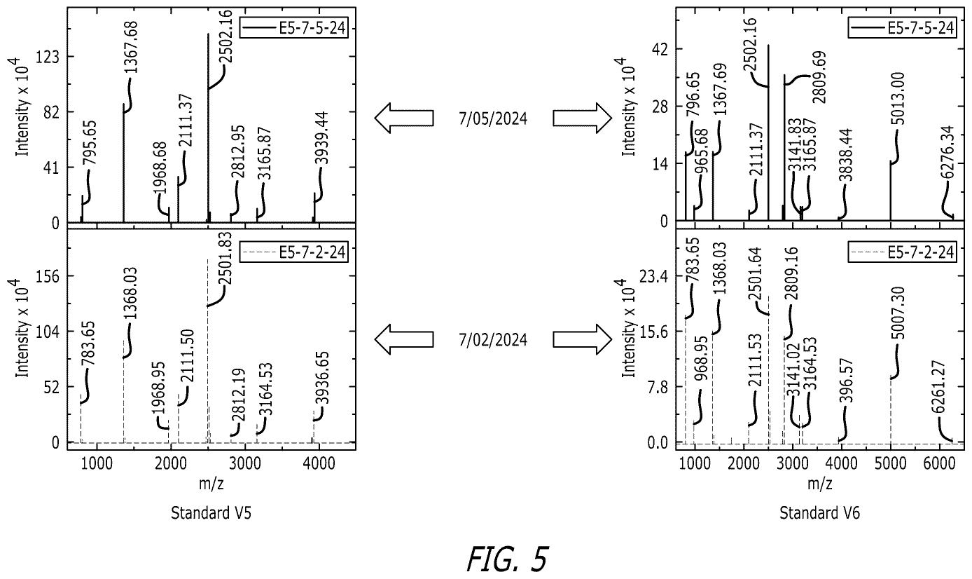

Stability of the molecular weight polypeptide standards under challenging environment conditions was assessed by repeating the initial MALDI-TOF analysis of Example 3 after exposing the polypeptides to harsh chemical compounds or repeated freeze-thaw cycles. In one series of experiments, exemplary molecular weight polypeptide standards V5 and V6 deposited on a MALDI plate in the presence of CHCA (α-cyano-4-hydroxycinnamic acid) matrix was stored at room temperature for three days. As shown in FIG. 5, an exemplary MALDI-TOF spectra analyzed using a linear positive mode indicated that exposure to CHCA after three days did not reveal detectable peptide deterioration indicating that stability of molecular weight polypeptide standards V5 and V6 was maintained even in the challenging environment of the MALDI matrix

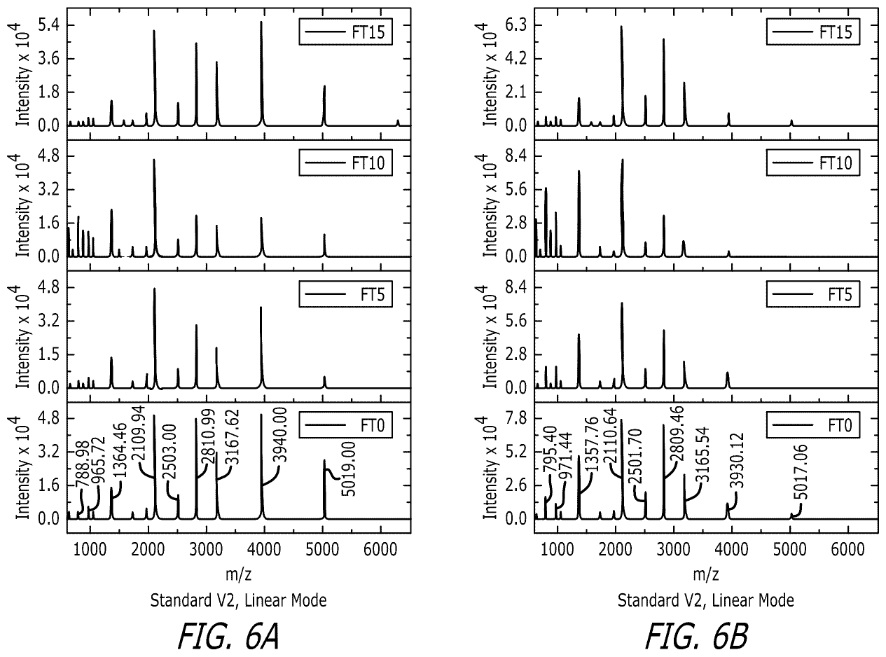

In another series of experiments, exemplary molecular weight polypeptide standards V2 underwent a series freeze-thaw cycles with spectral analysis conducts after 5, 10 and 15 cycles. Exemplary MALDI-TOF spectra analyzed using a linear positive mode (FIG. 6A) and a reflector positive mode (FIG. 6B) reveal that incubation of molecular weight polypeptide standards V2 did not lead to appreciable degradation of the peptides after 15 freeze-thaw cycles when compared to the control (0 freeze-thaw cycles) indicating that stability of molecular weight polypeptide standards V2 was maintained even after repeated freeze-thaw cycles.

In closing, foregoing descriptions of embodiments of the present invention have been presented for the purposes of illustration and description. It is to be understood that, although aspects of the present invention are highlighted by referring to specific embodiments, one skilled in the art will readily appreciate that these described embodiments are only illustrative of the principles comprising the present invention and such examples are not limiting thereto. As such, the specific embodiments are not intended to be exhaustive or to limit the invention to the precise forms disclosed. The use of any and all examples or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

In addition, groupings of alternative embodiments, elements, steps and/or limitations of the present invention are not to be construed as limitations. Each such grouping may be referred to and claimed individually or in any combination with other groupings disclosed herein. It is anticipated that one or more alternative embodiments, elements, steps and/or limitations of a grouping may be included in, or deleted from, the grouping for reasons of convenience and/or patentability. When any such inclusion or deletion occurs, the specification is deemed to contain the grouping as modified, thus fulfilling the written description of all Markush groups used in the appended claims. In addition, all methods described herein can be performed in any suitable order unless otherwise indicated herein or otherwise clearly contradicted by context. Therefore, it should be understood that embodiments of the disclosed subject matter are in no way limited to a particular element, compound, composition, component, article, apparatus, methodology, use, protocol, step, and/or limitation described herein, unless expressly stated as such.

While aspects of the inventive subject matter have been described with reference to at least one exemplary embodiment, it is to be clearly understood by those skilled in the art that the inventive subject matter is not limited thereto. For example, although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed. Furthermore, those of ordinary skill in the art will recognize that certain changes, modifications, permutations, alterations, additions, subtractions, and sub-combinations thereof can be made in accordance with the teachings herein without departing from the spirit of the present inventive subject matter. Thus, while the inventive subject matter is susceptible of various modifications and alternative embodiments, certain illustrated embodiments thereof are shown in the drawings and will be described below in detail. It should be understood, however, that there is no intention to limit the inventive subject matter to any specific form disclosed, but on the contrary, the inventive subject matter is to cover all modifications, alternative embodiments, and equivalents falling within the scope of the claims. It is intended that the following appended claims and claims hereafter introduced are interpreted to include all such changes, modifications, permutations, alterations, additions, subtractions, and sub-combinations as are within their true spirit and scope. Accordingly, the scope of the present inventive subject matter is not to be limited to that precisely as shown and described by this specification. Rather, the scope of the inventive subject matter is to be interpreted only in conjunction with the appended claims and it is made clear, here, that the inventor(s) believe that the claimed subject matter is the inventive subject matter.

Certain embodiments of the present inventive subject matter are described herein, including the best mode known to the inventors for conducting the inventive subject matter. Of course, variations on these described embodiments will become apparent to those of ordinary skill in the art upon reading the foregoing description. The inventor expects skilled artisans to employ such variations as appropriate, and the inventors intend for the present inventive subject matter to be practiced otherwise than specifically described herein. Accordingly, this inventive subject matter includes all modifications and equivalents of the subject matter recited in the claims appended hereto as permitted by applicable law. Moreover, any combination of the above-described embodiments in all possible variations thereof is encompassed by the inventive subject matter unless otherwise indicated herein or otherwise clearly contradicted by context.

The words, language, and terminology used in this specification is for the purpose of describing particular embodiments, elements, steps and/or limitations only and is not intended to limit the scope of the present inventive subject matter, which is defined solely by the claims. In addition, such words, language, and terminology are to be understood not only in the sense of their commonly defined meanings, but to include by special definition in this specification structure, material or acts beyond the scope of the commonly defined meanings. Thus, if an element, step or limitation can be understood in the context of this specification as including more than one meaning, then its use in a claim must be understood as being generic to all possible meanings supported by the specification and by the word itself.

The definitions and meanings of the elements, steps or limitations recited in a claim set forth below are, therefore, defined in this specification to include not only the combination of elements, steps or limitations which are literally set forth, but all equivalent structure, material or acts for performing substantially the same function in substantially the same way to obtain substantially the same result. In this sense it is therefore contemplated that an equivalent substitution of two or more elements, steps and/or limitations may be made for any one of the elements, steps or limitations in a claim set forth below or that a single element, step, or limitation may be substituted for two or more elements, steps and/or limitations in such a claim. Although elements, steps or limitations may be described above as acting in certain combinations and even initially claimed as such, it is to be expressly understood that one or more elements, steps and/or limitations from a claimed combination can in some cases be excised from the combination and that the claimed combination may be directed to a sub-combination or variation of a sub-combination. As such, notwithstanding the fact that the elements, steps and/or limitations of a claim are set forth below in a certain combination, it must be expressly understood that the inventive subject matter includes other combinations of fewer, more, or different elements, steps and/or limitations, which are disclosed in above combination even when not initially claimed in such combinations. Furthermore, insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements. Accordingly, the claims are thus to be understood to include what is specifically illustrated and described above, what is conceptually equivalent, what can be obviously substituted and also what essentially incorporates the essential idea of the inventive subject matter.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Similarly, as used herein, unless indicated to the contrary, the term “substantially” is a term of degree intended to indicate an approximation of the characteristic, item, quantity, parameter, property, or term so qualified, encompassing a range that can be understood and construed by those of ordinary skill in the art. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques.

Notwithstanding that the numerical ranges and values setting forth the broad scope of the inventive subject matter are approximations, the numerical ranges and values set forth in the specific examples are reported as precisely as possible. Any numerical range or value, however, inherently contains certain errors necessarily resulting from the standard deviation found in their respective testing measurements. Unless the context dictates the contrary, all ranges set forth herein should be interpreted as being inclusive of their endpoints and open-ended ranges should be interpreted to include only commercially practical values. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein. Similarly, all lists of values should be considered as inclusive of intermediate values unless the context indicates the contrary.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a comparable manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

The terms “a,” “an,” “the” and similar references used in the context of describing the present inventive subject matter (especially in the context of the following claims) are to be construed to cover both the singular and the plural, unless otherwise indicated herein or clearly contradicted by context. Further, ordinal indicators—such as, e.g., “first,” “second,” “third,” etc.—for identified elements are used to distinguish between the elements, and do not indicate or imply a required or limited number of such elements, and do not indicate a particular position or order of such elements unless otherwise specifically stated.

When used in the claims, whether as filed or added per amendment, the open-ended transitional term “comprising”, variations thereof such as, e.g., “comprise” and “comprises”, and equivalent open-ended transitional phrases thereof like “including”, “containing” and “having”, encompass all the expressly recited elements, limitations, steps, integers, and/or features alone or in combination with unrecited subject matter; the named elements, limitations, steps, integers, and/or features are essential, but other unnamed elements, limitations, steps, integers, and/or features may be added and still form a construct within the scope of the claim. Specific embodiments disclosed herein may be further limited in the claims using the closed-ended transitional phrases “consisting of” or “consisting essentially of” (or variations thereof such as, e.g., “consist of”, “consists of”, “consist essentially of”, and “consists essentially of”) in lieu of or as an amendment for “comprising.” When used in the claims, whether as filed or added per amendment, the closed-ended transitional phrase “consisting of” excludes any element, limitation, step, integer, or feature not expressly recited in the claims. The closed-ended transitional phrase “consisting essentially of” limits the scope of a claim to the expressly recited elements, limitations, steps, integers, and/or features and any other elements, limitations, steps, integers, and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps, integers, and/or features specifically recited in the claim, whereas the meaning of the closed-ended transitional phrase “consisting essentially of” is being defined as only including those elements, limitations, steps, integers, and/or features specifically recited in the claim and those elements, limitations, steps, integers, and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of.” As such, the embodiments described herein or so claimed with the phrase “comprising” expressly and unambiguously provide description, enablement, and support for the phrases “consisting essentially of” and “consisting of.”

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

Any claims intended to be treated under 35 U.S.C. § 112 (f) will begin with the words “means for,” but use of the term “for” in any other context is not intended to invoke treatment under 35 U.S.C. § 112 (f). Accordingly, Applicant reserves the right to pursue additional claims after filing this application, in either this application or in a continuing application.

It should be understood that the methods and the order in which the respective elements of each method are performed are purely exemplary. Depending on the implementation, they may be performed in any order or in parallel, unless indicated otherwise in the present disclosure.

Finally, all patents, patent publications, and other references cited and identified in the present specification are individually and expressly incorporated herein by reference in their entirety to the same extent as if each individual publication or patent application were specifically and individually indicated to be incorporated by reference. These publications are provided solely for their disclosure prior to the filing date of the present application. The reference to any prior art in this specification is not, and should not be taken as, an acknowledgement or any form of suggestion that the prior art forms part of the common general knowledge from any country. In addition, where a definition or use of a term in an incorporated reference is inconsistent or contrary to the definition of that term provided herein, the definition of that term provided herein applies and the definition of that term in the reference does not apply. Lastly, nothing in this regard is or should be construed as an admission that the inventors are not entitled to antedate such disclosure by virtue of prior invention or for any other reason. All statements as to the date or representation as to the contents of these documents are based on the information available to the applicant and do not constitute any admission as to the correctness of the dates or contents of these documents.

1. A recombinant protein comprising one or more repeating units, each of the one or more repeating units comprising a chemical cleavage site and a molecular weight polypeptide standard, wherein the chemical cleavage site is an amino acid sequence, the recombinant protein arranged according to formula I 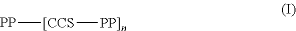
wherein
brackets indicate a repeating unit of the one or more repeating units;
PP is a molecular weight polypeptide standard;
CCS is a chemical cleavage site; and
n is an integer from 1 to 50.

2. The recombinant protein of claim 1, wherein the chemical cleavage site is a chemical cleavage site cleaved by a site-specific proteolytic chemical agent.

3. The recombinant protein of claim 2, wherein the site-specific proteolytic chemical agent is selected from 2-nitro-5-thiocyanobenzoic acid (NTCB), Ni²⁺, Cu²⁺, Co²⁺ or Pd²⁺.

4. The recombinant protein of claim 3, wherein the site-specific proteolytic chemical agent is Ni²⁺.

5. The recombinant protein of claim 1, wherein n is an integer from 5 to 25.

6. The recombinant protein of claim 1, wherein n is an integer from 5 to 15.

7. The recombinant protein of claim 1, wherein the repeating units comprise different molecular weight polypeptide standards, each having a determined molecular weight.

8. The recombinant protein of claim 6, wherein two or more of the different molecular weight polypeptide standards each have a different determined molecular weight.

9. The recombinant protein of claim 6, wherein two or more of the different molecular weight polypeptide standards each have the same determined molecular weight but a different amino acid sequence.

10. The recombinant protein of claim 1, wherein the recombinant protein comprises formula II or formula III: 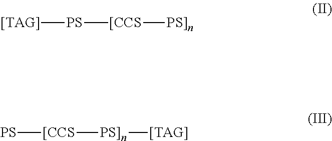
wherein
brackets indicate a repeating unit of the one or more repeating units;
PP is the product peptide;
CCS is the chemical cleavage site;
TAG is the affinity purification tag or the solubilization tag; and
n is an integer from 1 to 50.

11. The recombinant protein of claim 1, wherein the recombinant protein comprises formula IV or formula V: 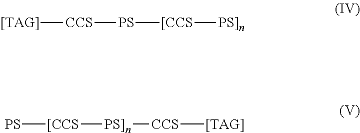
wherein
brackets indicate a repeating unit of the one or more repeating units;
PP is the product peptide;
CCS is the chemical cleavage site;
TAG is the affinity purification tag or the solubilization tag; and
n is an integer from 1 to 50.

12. The recombinant protein of claim 11, wherein the chemical cleavage site closest to the tag is replaced by a protease cleavage site.

13. The recombinant protein of claim 1, wherein the protein is storage stable in the presence of trifluoroacetic acid.

14. A nucleic acid encoding the recombinant polypeptide of claim 1.

15. An expression construct comprising an expression vector including the nucleic acid of claim 14.

16. An expression system comprising an expression construct of claim 15.

17. The expression system of claim 16, wherein the expression system is a cell-based system or a cell-free system.

18. A host cell comprising the expression construct of claim 15.

19. A method of producing a plurality of molecular weight polypeptide standards, the method comprising:
a) expressing a recombinant protein from a nucleic acid sequence encoding the recombinant protein, the recombinant protein comprising one or more repeating units, each of the one or more repeating units comprising a chemical cleavage site and a molecular weight polypeptide standard, wherein the chemical cleavage site is an amino acid sequence, the recombinant protein arranged according to formula I 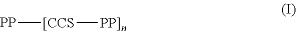
wherein
brackets indicate a repeating unit of the one or more repeating units;
PP is a molecular weight polypeptide standard;
CCS is a chemical cleavage site; and
n is an integer from 1 to 50;
b) isolating the expressed recombinant protein;
c) cleaving the chemical cleavage site or sites of the isolated recombinant protein using a chemical agent that recognizes the chemical cleavage site to produce a plurality of molecular weight polypeptide standards, wherein each molecular weight polypeptide standard has its amino acid sequence without any extraneous amino acids.

20. The method of claim 19, wherein the cleaving of step c) results in partial digestion of the recombinant protein or wherein the cleaving of step c) results in complete digestion of the polypeptide.


SEQUENCE LISTING (100 sequences)

SEQ ID NO: 1 - skipped

SEQ ID NO: 2 - protein, 5 aa, synthetic construct
  REGION 1..5: note = Enterokinase protease recognition site
DDDDK

SEQ ID NO: 3-4 - skipped (2 entries)

SEQ ID NO: 5 - protein, 4 aa, synthetic construct
  REGION 1..4: note = Caspase-1, -4, -5 recognition site
DEVD

SEQ ID NO: 6-15 - skipped (10 entries)

SEQ ID NO: 16 - protein, 4 aa, synthetic construct
  REGION 1..4: note = Factor Xa protease recognition site
IEGR

SEQ ID NO: 17 - protein, 4 aa, synthetic construct
  REGION 1..4: note = Factor Xa protease recognition site
IDGR

SEQ ID NO: 18-19 - skipped (2 entries)

SEQ ID NO: 20 - protein, 4 aa, synthetic construct
  REGION 1..4: note = Amino acid sequence of NTCB cleavage sequence
CGDE

SEQ ID NO: 21-27 - skipped (7 entries)

SEQ ID NO: 28 - protein, 4 aa, synthetic construct
  REGION 1..4: note = Amino acid sequence of Ni+ cleavage sequence
SRHW

SEQ ID NO: 29-38 - skipped (10 entries)

SEQ ID NO: 39 - protein, 5 aa, synthetic construct
  REGION 1..5: note = Amino acid sequence of linker
GGGGS

SEQ ID NO: 40-45 - skipped (6 entries)

SEQ ID NO: 46 - protein, 7 aa, synthetic construct
  REGION 1..7: note = Amino acid sequence of linker
GHHHHHH

SEQ ID NO: 47 - protein, 6 aa, synthetic construct
  REGION 1..6: note = Amino acid sequence of linker
EAAAKA

SEQ ID NO: 48 - protein, 6 aa, synthetic construct
  REGION 1..6: note = Amino acid sequence of linker
AEAAAK

SEQ ID NO: 49 - protein, 16 aa, synthetic construct
  REGION 1..16: note = Amino acid sequence of linker
KESGSVSSEQLAQFRS

SEQ ID NO: 50 - protein, 14 aa, synthetic construct
  REGION 1..14: note = Amino acid sequence of linker
EGKSSGSGSESKST

SEQ ID NO: 51 - protein, 12 aa, synthetic construct
  REGION 1..12: note = Amino acid sequence of linker
GSAGSAAGSGEF

SEQ ID NO: 52 - protein, 12 aa, synthetic construct
  REGION 1..12: note = Amino acid sequence of linker
GSAGSAAGSGEG

SEQ ID NO: 53 - protein, 31 aa, synthetic construct
  REGION 1..31: note = Amino acid sequence of linker
EAEDLQVGQVELGGGPGAGSLQPLALEGSLQ

SEQ ID NO: 54 - protein, 26 aa, synthetic construct
  REGION 1..26: note = Amino acid sequence of linker
GAGAGAMALWMRLLPLLALLALWGPD

SEQ ID NO: 55 - protein, 127 aa, synthetic construct
  REGION 1..127: note = Thioredoxin tag
MIAPILDEIADEYQGKLTVAKLNIDQNPGTAPKYGIRGIPTLLLFKNGEVAATKVGALSKGQLKEFLDANLAGSGSGHMHHHHHSSGLVPRGSGMKETAAAKFERQHMDSPDLGTDDDDKAMADIGS

SEQ ID NO: 56 - protein, 6 aa, synthetic construct
  REGION 1..6: note = His6 tag
HHHHHH

SEQ ID NO: 57 - protein, 26 aa, synthetic construct
  REGION 1..26: note = His tag
MHHHHHHALWMRLLPLLALLALWGPD

SEQ ID NO: 58 - protein, 313 aa, synthetic construct
  REGION 1..313: note = Recombinant polypeptide tag
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLGTDDDDKAMADIGSGDEVDHSQGTFTSDYSKYLDSRRAQDFVQWLMNTSRHWGDEVDHSQGTFTSDYSKYLDSRRAQDFVQWLMNTSRHWGDEVDHSQGTFTSDYSKYLDSRRAQDFVQWLMNTSRHWGDEVDHSQGTFTSDYSKYLDSRRAQDFVQWLMNT

SEQ ID NO: 59 - protein, 484 aa, synthetic construct
  REGION 1..484: note = Amino acid sequence of a MALDI recombinant polypeptide
MGSSHHHHHHSSGLVPRGSHMASMTGGQQMGRGSRHLLGSRHSPSLSASGALPVLSGSRHSLSLLGSRHSVSPPLSFSGSRHLLGSRHSLLSLGSRHSPVSSFPLSGSRHLLGSRHSVAPLGSASLLSPSGSRHSLLLSGSRHSPSLPSVFSGSRHSISGSSGVPRAPFAPGSVGSRHSIVFPSGSGPSVGAPRASGSRHSPSLLSFAPSGAVLSPFSVLSGSRHSGSLPSPLASLSFVALPSSFVGSRHSPSLLSFAPSGAVLSPFSVLSGSRHSALRVPVPLFGPALASLFAGSGFVGSRHSLPSGFAFVAAVLLRVAFSPGLGPGSRHSGAAAFFRSLPVGLVVAFSLPGLPGSRHSPALLPASVGAVAVFLFRSPFGLAAPGLGSRHSLSLAVVFSFPRAGAVFGPAALLAPLPGGSRHSLRVFPSAFFPLGVGLPASGLAALPAVAGSRHSPSGFVAVAPRLFAPVLFPSGLALAGALG

SEQ ID NO: 60 - protein, 30 aa, synthetic construct
  REGION 1..30: note = Amino acid sequence of a MALDI recombinant polypeptide
  MOD_RES 18: note = Oxidized methionine
  MOD_RES 21: note = Oxidized methionine
  MOD_RES 27: note = Oxidized methionine
SHHHHHHSSGLVPRGSHMASMTGGQQMGRG

SEQ ID NO: 61 - protein, 31 aa, synthetic construct
  REGION 1..31: note = Amino acid sequence of a MALDI recombinant polypeptide
  MOD_RES 19: note = Oxidized methionine
  MOD_RES 22: note = Oxidized methionine
  MOD_RES 28: note = Oxidized methionine
SSHHHHHHSSGLVPRGSHMASMTGGQQMGRG

SEQ ID NO: 62 - protein, 6 aa, synthetic construct
  REGION 1..6: note = Amino acid sequence of a MALDI recombinant polypeptide
SRHLLG

SEQ ID NO: 63 - protein, 6 aa, synthetic construct
  REGION 1..6: note = Amino acid sequence of a MALDI recombinant polypeptide
SRHLLG

SEQ ID NO: 64 - protein, 6 aa, synthetic construct
  REGION 1..6: note = Amino acid sequence of a MALDI recombinant polypeptide
SRHLLG

SEQ ID NO: 65 - protein, 9 aa, synthetic construct
  REGION 1..9: note = Amino acid sequence of a MALDI recombinant polypeptide
SRHSLSLLG

SEQ ID NO: 66 - protein, 9 aa, synthetic construct
  REGION 1..9: note = Amino acid sequence of a MALDI recombinant polypeptide
SRHSLLSLG

SEQ ID NO: 67 - protein, 9 aa, synthetic construct
  REGION 1..9: note = Amino acid sequence of a MALDI recombinant polypeptide
SRHSLLLSG

SEQ ID NO: 68 - protein, 13 aa, synthetic construct
  REGION 1..13: note = Amino acid sequence of a MALDI recombinant polypeptide
SRHSVSPPLSFSG

SEQ ID NO: 69 - protein, 13 aa, synthetic construct
  REGION 1..13: note = Amino acid sequence of a MALDI recombinant polypeptide
SRHSPVSSFPLSG

SEQ ID NO: 70 - protein, 13 aa, synthetic construct
  REGION 1..13: note = Amino acid sequence of a MALDI recombinant polypeptide
SRHSPSLPSVFSG

SEQ ID NO: 71 - protein, 18 aa, synthetic construct
  REGION 1..18: note = Amino acid sequence of a MALDI recombinant polypeptide
SRHSPSLSASGALPVLSG

SEQ ID NO: 72 - protein, 18 aa, synthetic construct
  REGION 1..18: note = Amino acid sequence of a MALDI recombinant polypeptide
SRHSVAPLGSASLLSPSG

SEQ ID NO: 73 - protein, 18 aa, synthetic construct
  REGION 1..18: note = Amino acid sequence of a MALDI recombinant polypeptide
SRHSPSLSASGALPVLSG

SEQ ID NO: 74 - protein, 22 aa, synthetic construct
  REGION 1..22: note = Amino acid sequence of a MALDI recombinant polypeptide
SRHSISGSSGVPRAPFAPGSVG

SEQ ID NO: 75 - protein, 22 aa, synthetic construct
  REGION 1..22: note = Amino acid sequence of a MALDI recombinant polypeptide
SRHSIVFPSGSGPSVGAPRASG

SEQ ID NO: 76 - protein, 25 aa, synthetic construct
  REGION 1..25: note = Amino acid sequence of a MALDI recombinant polypeptide
SRHSPSLLSFAPSGAVLSPFSVLSG

SEQ ID NO: 77 - protein, 25 aa, synthetic construct
  REGION 1..25: note = Amino acid sequence of a MALDI recombinant polypeptide
SRHSGSLPSPLASLSFVALPSSFVG

SEQ ID NO: 78 - protein, 25 aa, synthetic construct
  REGION 1..25: note = Amino acid sequence of a MALDI recombinant polypeptide
SRHSPSLLSFAPSGAVLSPFSVLSG

SEQ ID NO: 79 - protein, 28 aa, synthetic construct
  REGION 1..28: note = Amino acid sequence of a MALDI recombinant polypeptide
SRHSALRVPVPLFGPALASLFAGSGFVG

SEQ ID NO: 80 - protein, 28 aa, synthetic construct
  REGION 1..28: note = Amino acid sequence of a MALDI recombinant polypeptide
SRHSLPSGFAFVAAVLLRVAFSPGLGPG

SEQ ID NO: 81 - protein, 28 aa, synthetic construct
  REGION 1..28: note = Amino acid sequence of a MALDI recombinant polypeptide
SRHSGAAAFFRSLPVGLVVAFSLPGLPG

SEQ ID NO: 82 - protein, 32 aa, synthetic construct
  REGION 1..32: note = Amino acid sequence of a MALDI recombinant polypeptide
SRHSPALLPASVGAVAVFLFRSPFGLAAPGLG

SEQ ID NO: 83 - protein, 32 aa, synthetic construct
  REGION 1..32: note = Amino acid sequence of a MALDI recombinant polypeptide
SRHSLSLAVVFSFPRAGAVFGPAALLAPLPGG

SEQ ID NO: 84 - protein, 32 aa, synthetic construct
  REGION 1..32: note = Amino acid sequence of a MALDI recombinant polypeptide
SRHSLRVFPSAFFPLGVGLPASGLAALPAVAG

SEQ ID NO: 85 - protein, 32 aa, synthetic construct
  REGION 1..32: note = Amino acid sequence of a MALDI recombinant polypeptide
SRHSPSGFVAVAPRLFAPVLFPSGLALAGALG

SEQ ID NO: 86 - protein, 353 aa, synthetic construct
  REGION 1..353: note = Amino acid sequence of a MALDI recombinant polypeptide
MGSSHHHHHHSSGLVPRGSHMASMTGGQQMGRGSDEVDGSRHLLLGSRHSPSPSLFSVGSRHSISGSSGVPRAPFAPGSVGLSIVFPSGSGPSVGAPRASGSRHSPSLLSFAPSGAVLSPFSVLSGLRHSGSLPSPLASLSFVALPSSFVGSRHSPSLLSFAPSGAVLSPFSVLSGLHGSRHSPSLLSFAPSGAVLSPFSVLSGLHSGSLPSPLASLSFVALPSSFVGSRHSPALLPASVGAVAVFLFRSPFGLAAPGLGLSLSLAVVFSFPRAGAVFGPAALLAPLPGGSRHSLRVFPSAFFPLGVGLPASGLAALPAVAGLRHSPSGFVAVAPRLFAPVLFPSGLALAGAL

SEQ ID NO: 87 - protein, 36 aa, synthetic construct
  REGION 1..36: note = Amino acid sequence of a MALDI recombinant polypeptide
  MOD_RES 18: note = Oxidized methionine
  MOD_RES 21: note = Oxidized methionine
  MOD_RES 27: note = Oxidized methionine
SHHHHHHSSGLVPRGSHMASMTGGQQMGRGSDEVDG

SEQ ID NO: 88 - protein, 37 aa, synthetic construct
  REGION 1..37: note = Amino acid sequence of a MALDI recombinant polypeptide
  MOD_RES 19: note = Oxidized methionine
  MOD_RES 22: note = Oxidized methionine
  MOD_RES 28: note = Oxidized methionine
SSHHHHHHSSGLVPRGSHMASMTGGQQMGRGSDEVDG

SEQ ID NO: 89 - protein, 7 aa, synthetic construct
  REGION 1..7: note = Amino acid sequence of a MALDI recombinant polypeptide
SRHLLLG

SEQ ID NO: 90 - protein, 13 aa, synthetic construct
  REGION 1..13: note = Amino acid sequence of a MALDI recombinant polypeptide
SRHSPSPSLFSVG

SEQ ID NO: 91 - protein, 42 aa, synthetic construct
  REGION 1..42: note = Amino acid sequence of a MALDI recombinant polypeptide
SRHSISGSSGVPRAPFAPGSVGLSIVFPSGSGPSVGAPRASG

SEQ ID NO: 92 - protein, 50 aa, synthetic construct
  REGION 1..50: note = Amino acid sequence of a MALDI recombinant polypeptide
SRHSPSLLSFAPSGAVLSPFSVLSGLRHSGSLPSPLASLSFVALPSSFVG

SEQ ID NO: 93 - protein, 28 aa, synthetic construct
  REGION 1..28: note = Amino acid sequence of a MALDI recombinant polypeptide
SRHSPSLLSFAPSGAVLSPFSVLSGLHG

SEQ ID NO: 94 - protein, 49 aa, synthetic construct
  REGION 1..49: note = Amino acid sequence of a MALDI recombinant polypeptide
SRHSPSLLSFAPSGAVLSPFSVLSGLHSGSLPSPLASLSFVALPSSFVG

SEQ ID NO: 95 - protein, 62 aa, synthetic construct
  REGION 1..62: note = Amino acid sequence of a MALDI recombinant polypeptide
SRHSPALLPASVGAVAVFLFRSPFGLAAPGLGLSLSLAVVFSFPRAGAVFGPAALLAPLPGG

SEQ ID NO: 96 - protein, 63 aa, synthetic construct
  REGION 1..63: note = Amino acid sequence of a MALDI recombinant polypeptide
SRHSLRVFPSAFFPLGVGLPASGLAALPAVAGLRHSPSGFVAVAPRLFAPVLFPSGLALAGAL

SEQ ID NO: 97 - protein, 5 aa, synthetic construct
  REGION 1..5: note = Amino acid sequence of a pentapeptide
GKKGG

SEQ ID NO: 98 - protein, 5 aa, synthetic construct
  REGION 1..5: note = Amino acid sequence of a pentapeptide
GKGKG

SEQ ID NO: 99 - protein, 5 aa, synthetic construct
  REGION 1..5: note = Amino acid sequence of a pentapeptide
KGGGK

SEQ ID NO: 100 - protein, 4 aa, synthetic construct
  REGION 1..4: note = Amino acid sequence for a Ni2+ cleavage sequence
SRHS